(19) United States
(12) Patent Application Publication
(10) Pub. No.: US 2025/0297297 A1
(43) Pub. Date: Sep. 25, 2025

(54) APPARATUS AND METHOD TO SCREEN REAGENTS FOR IN VITRO TRANSCRIPTION

(71) Applicant: Nutcracker Therapeutics, Inc., Emeryville, CA (US)

(72) Inventors: Eric Chu, San Francisco, CA (US); Tamas Czimmermann, Hayward, CA (US); Benjamin Eldridge, Danville, CA (US); Kenneth Jordan, Danville, CA (US); Ximiao Wen, Hayward, CA (US); Ning Yu, Berkeley, CA (US)

(21) Appl. No.: 19/228,087

(22) Filed: Jun. 4, 2025

Related U.S. Application Data
(63) Continuation of application No. PCT/US2023/082233, filed on Dec. 4, 2023.
(60) Provisional application No. 63/430,139, filed on Dec. 5, 2022.

(51) Int. Cl. C12P 19/34; B01L 3/00; C12N 9/12; C12Q 1/6806

(52) U.S. Cl. CPC C12P 19/34; B01L 3/502715; C12N 9/1241; C12Q 1/6806

(57) ABSTRACT

A system includes a chip-receiving component, a first fluid processing assembly, a second fluid processing assembly, and a fluid communication pathway. The chip-receiving component is to receive a process chip having microfluidic passageways. The first fluid processing assembly is to communicate fluids to microfluidic passageways of a process chip received by the chip-receiving component. The second fluid processing assembly includes a sample support feature to support sample containers. The second fluid processing assembly also includes a plurality of sampling heads to selectively communicate fluids from sample containers supported by the sample support feature. The fluid communication pathway includes a plurality of conduits to provide fluid communication between the first fluid processing assembly and the plurality of sampling heads. The first fluid processing assembly is to further communicate fluids from the fluid communication pathway to microfluidic passageways of a process chip received by the chip-receiving component.

### BACKGROUND

The subject matter discussed in this section should not be assumed to be prior art merely as a result of its mention in this section. Similarly, a problem mentioned in this section or associated with the subject matter provided as background should not be assumed to have been previously recognized in the prior art. The subject matter in this section merely represents different approaches, which in and of themselves may also correspond to implementations of the claimed technology.

Some currently available technologies for manufacturing and formulating polynucleotide therapeutics (e.g., mRNA therapeutics, etc.) may expose the products to contamination and degradation. Some available centralized production may be too costly, too slow, or susceptible to contamination for use in therapeutic formulations possibly including multiple polynucleotide species.

### SUMMARY

Development of scalable polynucleotide manufacturing, production of single patient dosages, reduction, and in some instances even elimination, of touchpoints to limit contamination, input and process tracking for meeting clinical manufacturing requirements and use in point-of-care operations may advance the use of these therapeutic modalities. Microfluidic instrumentation and processes may provide advantages in achieving these goals. It may be desirable to facilitate rapid formulation of several samples of compositions, such as for screening purposes or otherwise. Described herein are devices, systems, and methods for facilitating rapid formulation of several samples of compositions through a microfluidic system, to overcome the pre-existing challenges and achieve the benefits as described herein. Such microfluidic systems may be used for the manufacture and formulation of biomolecule-containing products, such as therapeutics for individualized care.

An implementation relates to a system that includes a chip-receiving component, a first fluid processing assembly, a second fluid processing assembly, and a fluid communication pathway. The chip-receiving component is to receive a process chip having microfluidic passageways. The first fluid processing assembly is to communicate fluids to microfluidic passageways of a process chip received by the chip-receiving component. The second fluid processing assembly includes a sample support feature to support sample containers. The second fluid processing assembly also includes a plurality of sampling heads to selectively communicate fluids from sample containers supported by the sample support feature. The fluid communication pathway includes a plurality of conduits to provide fluid communication between the first fluid processing assembly and the plurality of sampling heads. The first fluid processing assembly is to further communicate fluids from the fluid communication pathway to microfluidic passageways of a process chip received by the chip-receiving component.

In some implementations of a system, such as that described in the preceding paragraph of this summary, the system further includes an instrument having a housing. The chip-receiving component and the first fluid processing assembly are positioned within the housing.

In some implementations of a system, such as any of those described in any of the preceding paragraphs of this summary, the second fluid processing assembly is positioned within the housing.

In some implementations of a system, such as any of those described in any of the preceding paragraphs of this summary, the system further includes a first controller to drive operation of the first fluid processing assembly.

In some implementations of a system, such as any of those described in any of the preceding paragraphs of this summary, the system further includes a second controller to drive operation of the second fluid processing assembly.

In some implementations of a system, such as any of those described in any of the preceding paragraphs of this summary, the second controller is in communication with the first controller.

In some implementations of a system, such as any of those described in any of the preceding paragraphs of this summary, the first controller is to drive operation of the second fluid processing assembly.

In some implementations of a system, such as any of those described in any of the preceding paragraphs of this summary, the chip-receiving component comprises a seating mount.

In some implementations of a system, such as any of those described in any of the preceding paragraphs of this summary, the first fluid processing assembly includes a reagent storage frame.

In some implementations of a system, such as any of those described in any of the preceding paragraphs of this summary, the first fluid processing assembly is to store one or more fluids.

In some implementations of a system, such as any of those described in any of the preceding paragraphs of this summary, the first fluid processing assembly is to store one or more reagents.

In some implementations of a system, such as any of those described in any of the preceding paragraphs of this summary, the first fluid assembly is to store one or more compositions created through a process chip received in the chip-receiving component.

In some implementations of a system, such as any of those described in any of the preceding paragraphs of this summary, the plurality of conduits include a plurality of flexible tubes.

In some implementations of a system, such as any of those described in any of the preceding paragraphs of this summary, the flexible tubes are removably coupled with one or both of the first fluid processing assembly or the second fluid processing assembly.

In some implementations of a system, such as any of those described in any of the preceding paragraphs of this summary, the sample support feature is to support a plurality of trays having a plurality of wells.

In some implementations of a system, such as any of those described in any of the preceding paragraphs of this summary, the sample support feature includes a plurality of tray indexing features, the tray indexing features to index the trays in relation to the sampling heads.

In some implementations of a system, such as any of those described in any of the preceding paragraphs of this summary, at least one of the tray indexing features is to resiliently bear against a tray.

In some implementations of a system, such as any of those described in any of the preceding paragraphs of this summary, the sample support feature is to support a first reagent tray to provide a first reagent. The sample support feature is to also support a composition tray to receive a composition formed using the process chip received by the chip-receiving component. The composition is formed using the first reagent.

In some implementations of a system, such as any of those described in any of the preceding paragraphs of this summary, the sample support feature is to support a second reagent tray to provide a second reagent, the composition tray to receive a composition formed using the process chip received by the chip-receiving component, the composition being formed using the first and second reagents.

In some implementations of a system, such as any of those described in any of the preceding paragraphs of this summary, the sample support feature is to support a rinse tray to provide a rinse fluid. The sample support feature is also to support a waste tray to receive waste generated through a rinsing process, the rinsing process including rinsing of one or more of the microfluidic passageways of a process chip received by the chip-receiving component.

In some implementations of a system, such as any of those described in any of the preceding paragraphs of this summary, the second fluid processing assembly further includes a sample support feature drive assembly to drive the sample support feature along one or more dimensions to position sample containers supported by the sample support feature in relation to the sampling heads.

In some implementations of a system, such as any of those described in any of the preceding paragraphs of this summary, the sample support feature drive assembly is to drive the sample support feature along two dimensions in a horizontal plane.

In some implementations of a system, such as any of those described in any of the preceding paragraphs of this summary, the second fluid processing assembly further includes a head support actuation assembly. The head support actuation assembly is to drive the sampling heads to position fluid-receiving portions of the sampling heads in fluids held by sample containers supported by the sample support feature.

In some implementations of a system, such as any of those described in any of the preceding paragraphs of this summary, the head support actuation assembly is to drive the sampling heads vertically to lower and raise the sampling heads in relation to the sample support feature.

In some implementations of a system, such as any of those described in any of the preceding paragraphs of this summary, each sampling head includes a body defining a first passageway. Each sampling head further includes a hollow shaft disposed in the first passageway of the body. The hollow shaft is to communicate fluid from a sample container supported by the sample support feature to the fluid communication pathway.

In some implementations of a system, such as any of those described in any of the preceding paragraphs of this summary, the first passageway has an inner diameter. The hollow shaft has an outer diameter. The outer diameter is less than the inner diameter such that a gap is defined between an outer surface of the hollow shaft and an inner surface of the first passageway.

In some implementations of a system, such as any of those described in any of the preceding paragraphs of this summary, the body further defines a lower opening in fluid communication with the gap. The body is to communicate pressurized air via the gap to the lower opening.

In some implementations of a system, such as any of those described in any of the preceding paragraphs of this summary, each sampling head further includes a pneumatic fitting and a second passageway. The second passageway and the pneumatic fitting are in fluid communication with the gap. The pneumatic fitting and the second passageway are to communicate pressurized air to the gap.

In some implementations of a system, such as any of those described in any of the preceding paragraphs of this summary, each sampling head is to drive fluid from a sample container supported by the sample support feature by communicating pressurized air to an interior region of the sample container.

In some implementations of a system, such as any of those described in any of the preceding paragraphs of this summary, each sampling head further includes a seal member to engage a portion of a sample container supported by the sample support feature.

In some implementations of a system, such as any of those described in any of the preceding paragraphs of this summary, the seal member comprises an annular gasket.

In some implementations of a system, such as any of those described in any of the preceding paragraphs of this summary, the system further includes a biasing member to resiliently urge the seal member into engagement with the portion of a sample container supported by the sample support feature.

In some implementations of a system, such as any of those described in any of the preceding paragraphs of this summary, the second fluid processing assembly further includes a sample container engagement assembly to selectively engage a sample container supported by the sample support feature.

In some implementations of a system, such as any of those described in any of the preceding paragraphs of this summary, the sample container engagement assembly includes a foot and one or more actuators to selectively drive the foot into and out of engagement with a sample container supported by the sample support feature.

In some implementations of a system, such as any of those described in any of the preceding paragraphs of this summary, the sample support feature includes a platform.

Another implementation relates to an apparatus that includes a sample support feature to support sample containers, a sampling head assembly, and a head support actuation assembly. The sampling head assembly includes amounting body and a plurality of sampling heads supported by the mounting body. Each sampling head includes a sampling head body and a hollow shaft supported by the sampling head body. The hollow shaft includes a lower end to receive fluid from a sample container supported by the sample support feature. Each sampling head further includes a seal member to seal against a surface of a sample container supported by the sample support feature. Each sampling head further includes an opening to communicate pressurized air into a space defined above a volume of fluid in a sample container supported by the sample support feature to thereby drive the fluid from the sample container into the hollow shaft. The head support actuation assembly includes a head support plate and one or more actuators. The mounting body is mounted to the head support plate. The one or more actuators are to drive the head support plate toward the sample support feature to thereby selectively urge the lower ends of the hollow shafts into a sample container supported by the sample support feature.

In some implementations of an apparatus, such as that described in the preceding paragraph of this summary, the apparatus further includes a plurality of fluid conduits to couple the sampling head assembly with a fluid processing assembly to thereby communicate fluid from a sample container supported by the sample support feature to microfluidic channels in a process chip via the fluid processing assembly.

In some implementations of an apparatus, such as any of those described in any of the preceding paragraphs of this summary, the apparatus further includes a sample support feature drive assembly to drive the sample support feature along one or more dimensions to position sample containers supported by the sample support feature in relation to the sampling head assembly.

In some implementations of an apparatus, such as any of those described in any of the preceding paragraphs of this summary, the sample support feature drive assembly is to drive the sample support feature along two dimensions in a horizontal plane.

In some implementations of an apparatus, such as any of those described in any of the preceding paragraphs of this summary, the sampling head body defining a first passageway, the hollow shaft is disposed in the first passageway of the sampling head body.

In some implementations of an apparatus, such as any of those described in any of the preceding paragraphs of this summary, the first passageway has an inner diameter. The hollow shaft has an outer diameter. The outer diameter is less than the inner diameter such that a gap is defined between an outer surface of the hollow shaft and an inner surface of the first passageway.

In some implementations of an apparatus, such as any of those described in any of the preceding paragraphs of this summary, the opening of the sampling head assembly is in fluid communication with the gap. The sampling head body is to communicate pressurized air via the gap to the opening.

In some implementations of an apparatus, such as any of those described in any of the preceding paragraphs of this summary, each sampling head further includes a pneumatic fitting and a second passageway. The second passageway and the pneumatic fitting are in fluid communication with the gap. The pneumatic fitting and the second passageway are to communicate pressurized air to the gap.

In some implementations of an apparatus, such as any of those described in any of the preceding paragraphs of this summary, the seal member comprises an annular gasket.

In some implementations of an apparatus, such as any of those described in any of the preceding paragraphs of this summary, each sampling head assembly further includes a resilient member to resiliently urge the seal into engagement with the surface of a sample container supported by the sample support feature.

In some implementations of an apparatus, such as any of those described in any of the preceding paragraphs of this summary, the resilient member is interposed between a portion of the mounting body and the sampling head body.

In some implementations of an apparatus, such as any of those described in any of the preceding paragraphs of this summary, the resilient member is to compress to thereby accommodate a range of vertical motion of the sampling head body in relation to the mounting body.

In some implementations of an apparatus, such as any of those described in any of the preceding paragraphs of this summary, the apparatus further includes a sample container engagement assembly to selectively engage a sample container supported by the sample support feature.

In some implementations of an apparatus, such as any of those described in any of the preceding paragraphs of this summary, sample container engagement assembly includes a foot and one or more actuators to selectively drive the foot into and out of engagement with a sample container supported by the sample support feature.

In some implementations of an apparatus, such as any of those described in any of the preceding paragraphs of this summary, the one or more actuators are secured to the head support plate.

In some implementations of an apparatus, such as any of those described in any of the preceding paragraphs of this summary, the foot defines a plurality of openings. Each opening is configured to accommodate a corresponding hollow shaft of the plurality of sampling heads.

Another implementation relates to an apparatus that includes a sample support feature to support sample containers, a sampling head assembly, a head support actuation assembly, and a sample container engagement assembly. The sampling head assembly includes a mounting body and a plurality of sampling heads supported by the mounting body. Each sampling head includes a sampling head body and a hollow shaft supported by the sampling head body. The hollow shaft includes a lower end to receive fluid from a sample container supported by the sample support feature. The head support actuation assembly includes a head support plate and one or more actuators. The mounting body is mounted to the head support plate. The one or more actuators are to drive the head support plate toward the sample support feature to thereby selectively urge the lower ends of the hollow shafts into a sample container supported by the sample support feature. The sample container engagement assembly is to selectively engage a sample container supported by the sample support feature. The sample container engagement assembly includes a foot and one or more actuators to selectively drive the foot into and out of engagement with a sample container supported by the sample support feature.

In some implementations of an apparatus, such as that described in the preceding paragraph of this summary, the one or more actuators are secured to the head support plate.

In some implementations of an apparatus, such as any of those described in any of the preceding paragraphs of this summary, the foot defines a plurality of openings. Each opening is configured to accommodate a corresponding hollow shaft of the plurality of sampling heads.

In some implementations of an apparatus, such as any of those described in any of the preceding paragraphs of this summary, the apparatus further includes a plurality of fluid conduits to couple the sampling head assembly with a fluid processing assembly to thereby communicate fluid from a sample container supported by the sample support feature to microfluidic channels in a process chip via the fluid processing assembly.

In some implementations of an apparatus, such as any of those described in any of the preceding paragraphs of this summary, the apparatus further includes a sample support feature drive assembly to drive the sample support feature along one or more dimensions to position sample containers supported by the sample support feature in relation to the sampling head assembly.

In some implementations of an apparatus, such as any of those described in any of the preceding paragraphs of this summary, the sample support feature drive assembly is to drive the sample support feature along two dimensions in a horizontal plane.

In some implementations of an apparatus, such as any of those described in any of the preceding paragraphs of this summary, the sampling head body defines a first passageway. The hollow shaft is disposed in the first passageway of the sampling head body.

In some implementations of an apparatus, such as any of those described in any of the preceding paragraphs of this summary, the first passageway has an inner diameter. The hollow shaft has an outer diameter. The outer diameter is less than the inner diameter such that a gap is defined between an outer surface of the hollow shaft and an inner surface of the first passageway.

In some implementations of an apparatus, such as any of those described in any of the preceding paragraphs of this summary, the opening of the sampling head assembly is in fluid communication with the gap. The sampling head body is to communicate pressurized air via the gap to the opening.

In some implementations of an apparatus, such as any of those described in any of the preceding paragraphs of this summary, each sampling head further includes a pneumatic fitting and a second passageway. The second passageway and the pneumatic fitting are in fluid communication with the gap. The pneumatic fitting and the second passageway are to communicate pressurized air to the gap.

In some implementations of an apparatus, such as any of those described in any of the preceding paragraphs of this summary, each sampling head further includes a seal member and an opening. The seal member is to seal against a surface of a sample container supported by the sample support feature. The opening is to communicate pressurized air into a space defined above a volume of fluid in a sample container supported by the sample support feature to thereby drive the fluid from the sample container into the hollow shaft.

In some implementations of an apparatus, such as any of those described in any of the preceding paragraphs of this summary, the seal member comprises an annular gasket.

In some implementations of an apparatus, such as any of those described in any of the preceding paragraphs of this summary, each sampling head assembly further includes a resilient member to resiliently urge the seal into engagement with the surface of a sample container supported by the sample support feature.

In some implementations of an apparatus, such as any of those described in any of the preceding paragraphs of this summary, the resilient member is interposed between a portion of the mounting body and the sampling head body.

In some implementations of an apparatus, such as any of those described in any of the preceding paragraphs of this summary, the resilient member is to compress to thereby accommodate a range of vertical motion of the sampling head body in relation to the mounting body.

Another implementation relates to a method that includes positioning a plurality of sampling heads over a plurality of fluid containers. The method further includes inserting hollow shafts of the sampling heads into the plurality of fluid containers. The method further includes driving a first reagent from a first subset of the fluid containers via a first subset of the hollow shafts toward microfluidic passageways in a process chip. The method further includes driving a second reagent toward microfluidic passageways in the process chip. The method further includes combining the first and second reagent via the process chip to form a composition. The method further includes driving the composition from the process chip to a second subset of the fluid containers via a second subset of the hollow shafts.

In some implementations of a method, such as that described in the preceding paragraph of this summary, the second reagent is contained in a third subset of the fluid containers. The driving the second reagent toward microfluidic passageways in the process chip includes driving the second reagent from the third subset of the fluid containers via a third subset of the hollow shafts.

In some implementations of a method, such as any of those described in any of the preceding paragraphs of this summary, the method further includes driving a buffer toward microfluidic passageways in the process chip. The combining the first and second reagent via the process chip to form a composition includes combining the buffer with the first reagent.

In some implementations of a method, such as any of those described in any of the preceding paragraphs of this summary, the first reagent includes an mRNA.

In some implementations of a method, such as any of those described in any of the preceding paragraphs of this summary, the second reagent includes a delivery vehicle.

In some implementations of a method, such as any of those described in any of the preceding paragraphs of this summary, the composition includes an encapsulated mRNA.

In some implementations of a method, such as any of those described in any of the preceding paragraphs of this summary, the encapsulated mRNA includes mRNA encapsulated in delivery vehicle molecules having a geometry of a nanoparticle.

In some implementations of a method, such as any of those described in any of the preceding paragraphs of this summary, the method further includes priming the microfluidic passageways in the process chip.

In some implementations of a method, such as any of those described in any of the preceding paragraphs of this summary, the priming includes monitoring target areas in the microfluidic passageways in the process chip. The priming further includes detecting a presence of fluid in the target areas. The priming further includes arresting movement of fluid in the microfluidic passageways in response to detecting the presence of fluid in the target areas.

In some implementations of a method, such as any of those described in any of the preceding paragraphs of this summary, the method further includes removing the hollow shafts of the sampling heads from the plurality of fluid containers.

In some implementations of a method, such as any of those described in any of the preceding paragraphs of this summary, the method further includes engaging the plurality of fluid containers with a foot before inserting hollow shafts of the sampling heads into the plurality of fluid containers. The method further includes releasing the plurality of fluid containers from the foot after removing the hollow shafts of the sampling heads from the plurality of fluid containers.

In some implementations of a method, such as any of those described in any of the preceding paragraphs of this summary, the method further includes rinsing the hollow shafts and the microfluidic passageways in the process chip after driving the composition from the process chip to a second subset of the fluid containers via the second subset of the hollow shafts.

In some implementations of a method, such as any of those described in any of the preceding paragraphs of this summary, the method further includes drying the hollow shafts and the microfluidic passageways in the process chip after rinsing the hollow shafts and the microfluidic passageways in the process chip.

In some implementations of a method, such as any of those described in any of the preceding paragraphs of this summary, the method further includes collecting a rinse waste fluid in a third subset of the fluid containers. The rinse waste fluid is generated by rinsing the hollow shafts and the microfluidic passageways in the process chip.

In some implementations of a method, such as any of those described in any of the preceding paragraphs of this summary, the method further includes determining whether another subset of the fluid containers contains more of the first reagent.

In some implementations of a method, such as any of those described in any of the preceding paragraphs of this summary, the method further includes determining that a third subset of the fluid containers contains more of the first reagent. The method further includes positioning a plurality of sampling heads over the third subset of fluid containers. The method further includes inserting hollow shafts of the sampling heads into the third subset of fluid containers. The method further includes driving a first reagent from the third subset of the fluid containers via the first subset of the hollow shafts toward microfluidic passageways in a process chip. The method further includes driving a second reagent toward microfluidic passageways in the process chip. The method further includes combining the first and second reagent via the process chip to form a subsequent composition. The method further includes driving the subsequent composition from the process chip to a fourth subset of the fluid containers via the second subset of the hollow shafts.

In some implementations of a method, such as any of those described in any of the preceding paragraphs of this summary, the method further includes determining that another subset of the fluid containers does not contain more of the first reagent. The method further includes alerting a user that a composition forming process is complete.

Another implementation relates to a processor-readable medium including contents that are to cause a processor to process data by performing a method such as any of those described in any of the preceding paragraphs of this summary.

Another implementation relates to a method that includes communicating a first template to a process chip, the first template being located in a first well of a first tray. The method further includes communicating a first set of one or more reagents to the process chip. The method further includes performing in vitro transcription (IVT) utilizing the first template and the first set of one or more reagents, thereby creating a first RNA solution. The method further includes communicating the first RNA solution to a first well of a second tray. The method further includes communicating a second template to the process chip, the second template being located in a second well of the first tray. The method further includes communicating a second set of one or more reagents to the process chip. The method further includes performing in vitro transcription (IVT) utilizing the second template and the second set of one or more reagents, thereby creating a second RNA solution. The method further includes communicating the second RNA solution to a second well of the second tray.

In some implementations of a method, that described in the preceding paragraph of this summary, the one or more reagents being contained in one or more corresponding vials.

In some implementations of a method, such as any of those described in any of the preceding paragraphs of this summary, the method further includes communicating a cleaning agent through the process chip between the act of communicating the first RNA solution to the first well of the second tray and communicating the second template to the process chip.

In some implementations of a method, such as any of those described in any of the preceding paragraphs of this summary, the cleaning agent comprising a component selected from the group consisting of tris(hydroxymethyl)aminomethane, sodium hydroxide, sodium hypochlorite, polysorbate, water, and combinations thereof.

In some implementations of a method, such as any of those described in any of the preceding paragraphs of this summary, the cleaning agent is communicated from one or more wells of a third tray.

In some implementations of a method, such as any of those described in any of the preceding paragraphs of this summary, the method further includes communicating a rinsing agent through the process chip between the act of communicating the first RNA solution to the first well of the second tray and communicating the second template to the process chip.

In some implementations of a method, such as any of those described in any of the preceding paragraphs of this summary, the rinsing agent comprising water.

In some implementations of a method, such as any of those described in any of the preceding paragraphs of this summary, the rinsing agent is communicated from one or more wells of a third tray.

In some implementations of a method, such as any of those described in any of the preceding paragraphs of this summary, the method further includes automatically priming the process chip with the first template and the first set of one or more reagents.

In some implementations of a method, such as any of those described in any of the preceding paragraphs of this summary, the first set of one or more reagents includes one or more components selected from the group of NTPs, polymerase, and buffer.

In some implementations of a method, such as any of those described in any of the preceding paragraphs of this summary, the first template includes luciferase or green-fluorescent protein.

In some implementations of a method, such as any of those described in any of the preceding paragraphs of this summary, the method further includes testing the first RNA solution. The method further includes testing the second RNA solution. The method further includes determining which of the first RNA solution and the second RNA solution provides a best fit with one or more criteria.

In some implementations of a method, such as any of those described in any of the preceding paragraphs of this summary, the method further includes transporting the second tray to a testing location to perform the acts of testing the first RNA solution and testing the second RNA solution.

Another implementation relates to a processor-readable medium including contents that are to cause a processor to process data by performing a method such as any of those described in any of the preceding paragraphs of this summary.

Another implementation relates to a system. The system includes a first fluid processing assembly. The first fluid processing assembly includes a process chip. The system further includes a second fluid processing assembly. The second fluid processing assembly includes a first tray and a second tray. The system further includes one or more controllers that are configured to execute a control algorithm to automatically perform a process. The performed process includes communicating a first template to the process chip, the first template being located in a first well of the first tray. The performed process further includes communicating a first set of one or more reagents to the process chip. The performed process further includes performing in vitro transcription (IVT) utilizing the first template and the first set of one or more reagents, thereby creating a first RNA solution. The performed process further includes communicating the first RNA solution to a first well of the second tray. The performed process further includes communicating a second template to the process chip, the second template being located in a second well of the first tray. The performed process further includes communicating a second set of one or more reagents to the process chip. The performed process further includes performing in vitro transcription (IVT) utilizing the second template and the second set of one or more reagents, thereby creating a second RNA solution. The performed process further includes communicating the second RNA solution to a second well of the second tray.

It should be appreciated that all combinations of the foregoing concepts and additional concepts discussed in greater detail below (provided such concepts are not mutually inconsistent) are contemplated as being part of the inventive subject matter disclosed herein and to achieve the benefits as described herein.

### BRIEF DESCRIPTION OF THE DRAWINGS

The details of one or more implementations are set forth in the accompanying drawings and the description below. Other features, aspects, and advantages will become apparent from the description, the drawings, and the claims, in which:

FIG. 1 depicts a schematic view of an example of a system including a microfluidic process chip;

FIG. 2 depicts an exploded perspective view of examples of components of the system of FIG. 1;

FIG. 3 depicts a top plan view of an example of a process chip that may be incorporated into the system of FIG. 1;

FIG. 4 schematically illustrates an example of a method of manufacturing an mRNA therapeutic composition;

FIG. 5 shows a top plan view of examples of mixing stages that may be incorporated into a process chip that is used for formulation of mRNA with a delivery vehicle;

FIG. 6 depicts a schematic view of an example of a system including an instrument for processing fluids on a process chip and an additional fluid processing subsystem;

FIG. 7 depicts a schematic view of an example of a system including an instrument with a first fluid processing assembly and a second fluid processing assembly;

FIG. 8 depicts a schematic view of an example of a system that may be used to prepare several samples of compositions;

FIG. 9 depicts a perspective view of an example of a fluid processing assembly

FIG. 10A depicts a top plan view of the fluid processing assembly of FIG. 9, with a tray support platform in a first position;

FIG. 10B depicts a top plan view of the fluid processing assembly of FIG. 9, with the tray support platform in a second position;

FIG. 10C depicts a top plan view of the fluid processing assembly of FIG. 9, with the tray support platform in a third position;

FIG. 11A depicts a side elevation view of the fluid processing assembly of FIG. 9, with a head support plate in a first position;

FIG. 11B depicts a side elevation view of the fluid processing assembly of FIG. 9, with the head support plate in a second position;

FIG. 12 depicts a perspective view of the tray support platform of the fluid processing assembly of FIG. 9, loaded with a plurality of trays;

FIG. 13A depicts a top plan view of a portion of the tray support platform of FIG. 12, before a tray is loaded on the tray support platform;

FIG. 13B depicts a top plan view of the portion of the tray support platform of FIG. 13A, with a tray on the tray support platform in a non-indexed position;

FIG. 13C depicts a top plan view of the portion of the tray support platform of FIG. 13A, with a tray on the tray support platform in an indexed position;

FIG. 14 depicts a side elevation view of a portion of the tray support platform of FIG. 12, with an alignment feature maintaining the tray in the indexed position;

FIG. 15 depicts a perspective view of an example of a sampling head assembly of the fluid processing assembly of FIG. 9, with a sampling head disassembled from a body of the sampling head assembly;

FIG. 16 depicts an exploded perspective view of a sampling head of the sampling head assembly of FIG. 15;

FIG. 17 depicts a cross-sectional side view of a body of the sampling head of FIG. 16;

FIG. 18 depicts a cross-sectional side view of a lower portion of the sampling head of FIG. 16;

FIG. 19 depicts a cross-sectional side view of an upper portion of the sampling head of FIG. 16;

FIG. 20 depicts a perspective view of a tray engagement assembly of the fluid processing assembly of FIG. 9;

FIG. 21 depicts another perspective view of the tray engagement assembly of FIG. 20;

FIG. 22A depicts a side elevational view of the head support plate, sampling head assembly, and tray engagement assembly of the fluid processing assembly of FIG. 9 in a first operational state in relation to a tray;

FIG. 22B depicts a side elevational view of the components of FIG. 22A in a second operational state in relation to the tray;

FIG. 22C depicts a side elevational view of the components of FIG. 22A in a third operational state in relation to the tray;

FIG. 22D depicts a side elevational view of the components of FIG. 22A in a fourth operational state in relation to the tray;

FIG. 22E depicts a side elevational view of the components of FIG. 22A in a fifth operational state in relation to the tray;

FIG. 22F depicts a side elevational view of the components of FIG. 22A in a sixth operational state in relation to the tray;

FIG. 22G depicts a side elevational view of the components of FIG. 22A in a seventh operational state in relation to the tray;

FIG. 23 depicts a flow chart of an example of a method that may be performed using the fluid processing assembly of FIG. 9;

FIG. 24 depicts a schematic view of an example of a system that may be used to perform several in vitro transcription processes using different templates;

FIG. 25 depicts a flow chart of an example of a method that may be used to set up the system of FIG. 24; and

FIG. 26 depicts a flow chart of an example of a method that may be used to perform several in vitro transcription processes using different templates using the system of FIG. 24.

### DETAILED DESCRIPTION

In some aspects, apparatuses and methods are disclosed herein for processing therapeutic polynucleotides. In particular, these apparatuses and methods may be closed path apparatuses and methods that are configured to minimize or eliminate manual handling during operation. The closed path apparatuses and methods may provide a nearly entirely aseptic environment, and the components may provide a sterile path for processing from initial input (e.g., template) to output (e.g., compounded therapeutic). Material inputs (e.g., nucleotides, and any chemical components) into the apparatus may be sterile; and may be input into the system without requiring virtually any manual interaction.

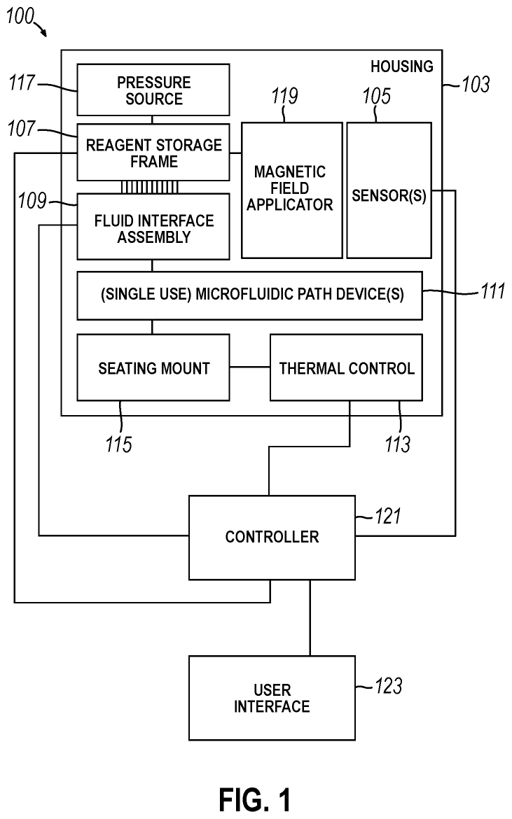

The apparatuses and methods described herein may be used to generate therapeutics at rapid cycle times at high degree of reproducibility. The apparatuses described herein may be configured to provide, in a single integrated apparatus, synthesis, purification, dialysis, compounding, and concentration of one or more therapeutic compositions. Alternatively, one or more of these processes may be carried out in two or more apparatuses as described herein. In some scenarios, the therapeutic compositions may include therapeutic polynucleotides, such as, for example, ribonucleic acids or deoxyribonucleic acids. The polynucleotides may include only natural nucleotide units or may include any kind of synthetic, semi-synthetic, or modified nucleotide units. All or some of the processing steps may be performed in an unbroken fluid processing pathway, which may be configured as one or a series of consumable microfluidic path device(s)—in some instances also referred to herein as a process chip or a biochip (though the chip need not necessarily be used in bio-related applications). The process chip in in some examples may be removably installed in an instrument that is part of a larger microfluidic system, such as that shown in FIG. 1). The disclosed apparatuses and methods may be used for the synthesis of patient-specific therapeutics, including compounding, at a point of care (e.g., hospital, clinic, pharmacy, etc.).

### I. Terminology

Throughout this specification and the claims which follow, unless the context requires otherwise, the word “comprise,” and variations such as “comprises” and “comprising” means various components may be co-jointly employed in the methods and articles (e.g., compositions and apparatuses including device and methods). For example, the term “comprising” will be understood to imply the inclusion of any stated elements or steps but not the exclusion of any other elements or steps. In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components, or sub-steps.

As used herein, the singular forms “a,” “an,” and “the” are intended to include the plural forms as well, unless the context clearly indicates otherwise. As used herein, the term “and/or” includes any and all combinations of one or more of the associated listed items and may be abbreviated as “/”.

Spatially relative terms, such as “under,” “below,” “lower,” “over,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the term “under” may encompass both an orientation of over and under. The device may be otherwise oriented (rotated 90 degrees or at other orientations) and the spatially relative descriptors used herein interpreted accordingly. Similarly, the terms “upwardly,” “downwardly,” “vertical,” “horizontal,” and the like are used herein for the purpose of explanation only unless specifically indicated otherwise.

When a feature or element is herein referred to as being “on” another feature or element, it may be directly on the other feature or element or intervening features and/or elements may also be present. In contrast, when a feature or element is referred to as being “directly on” another feature or element, there are no intervening features or elements present. When a feature or element is referred to as being “connected,” “attached,” or “coupled” to another feature or element, it may be directly connected, attached, or coupled to the other feature or element or intervening features or elements may be present. In contrast, when a feature or element is referred to as being “directly connected,” “directly attached,” or “directly coupled” to another feature or element, there are no intervening features or elements present. Although described or shown with respect to one embodiment, the features and elements so described or shown may apply to other embodiments. It will also be appreciated by those skilled in the art that references to a structure or feature that is disposed “adjacent” another feature may have portions that overlap or underlie the adjacent feature.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is ±0.1% of the stated value (or range of values), ±1% of the stated value (or range of values), ±2% of the stated value (or range of values), ±5% of the stated value (or range of values), ±10% of the stated value (or range of values), etc. Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein.

It is also understood that when a value is disclosed that “less than or equal to” the value, “greater than or equal to the value,” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although the terms “first” and “second” may be used herein to describe various features/elements (including steps), these features/elements should not be limited by these terms, unless the context indicates otherwise. These terms are used to distinguish one feature/element from another feature/element, and unless specifically pointed out, do not denote a certain order. Thus, a first feature/element discussed below could be termed a second feature/element, and similarly, a second feature/element discussed below could be termed a first feature/element without departing from the teachings of the present invention.

As used herein, the terms “system,” “apparatus,” and “device” may be read as being interchangeable with each other. A system, apparatus, and device may each include a plurality of components having various kinds of structural and/or functional relationships with each other.

As used herein, “polynucleotide” refers to a nucleic acid molecule containing multiple nucleotides and generally refers both to “oligonucleotides” (a polynucleotide molecule of 18-25 nucleotides in length) and polynucleotides of 26 or more nucleotides. Aspects of this disclosure include compositions including oligonucleotides having a length of 18-25 nucleotides (e.g., 18-mers, 19-mers, 20-mers, 21-mers, 22-mers, 23-mers, 24-mers, or 25-mers), or medium-length polynucleotides having a length of 26 or more nucleotides (e.g., polynucleotides of 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, or about 300 nucleotides), or long polynucleotides having a length greater than about 300 nucleotides (e.g., polynucleotides of between about 300 to about 400 nucleotides, between about 400 to about 500 nucleotides, between about 500 to about 600 nucleotides, between about 600 to about 700 nucleotides, between about 700 to about 800 nucleotides, between about 800 to about 900 nucleotides, between about 900 to about 1000 nucleotides, between about 300 to about 500 nucleotides, between about 300 to about 600 nucleotides, between about 300 to about 700 nucleotides, between about 300 to about 800 nucleotides, between about 300 to about 900 nucleotides, or about 1000 nucleotides in length, or even greater than about 1000 nucleotides in length. Where a polynucleotide is double-stranded, its length may be similarly described in terms of base pairs.

As used herein “amplification” may refer to polynucleotide amplification. Amplification may include any suitable method for amplification of a polynucleotide and includes, but is not limited to, multiple displacement amplification (MDA), polymerase chain reaction (PCR) amplification, Loop Mediated Isothermal Amplification (LAMP), Nucleic Acid Sequence Based Amplification, Strand Displacement Amplification, Rolling Circle Amplification, and Ligase Chain Reaction.

As used herein a “cassette” (e.g., a synthetic in vitro transcription facilitator cassette) refers to a polynucleotide sequence which may include or be operably linked to one or more expression elements such as an enhancer, a promoter, a leader, an intron, a 5′ untranslated region (UTR), a 3′ UTR, or a transcription termination sequence. In some aspects, a cassette comprises at least a first polynucleotide sequence capable of initiating transcription of an operably linked second polynucleotide sequence (which may comprise a template) and optionally a transcription termination sequence operably linked to the second polynucleotide sequence. The template, as described below, may comprise a sequence of interest, for example, an open reading frame (“ORF”) of interest. The cassette may be provided as a single element or as two or more unlinked elements.

As used herein, a “template” refers to a nucleic acid sequence that contains a sequence of interest for preparing a therapeutic polynucleotide according to the disclosed methods. Templates may be, but are not limited to, a double stranded DNA (dsDNA), an engineered plasmid construct, a cDNA sequence, or a linear nucleic acid sequence (for example, a linear template generated by PCR or by annealing chemically synthesized oligonucleotides). The template may, in certain aspects, be integrated into a “cassette” as described above.

As used herein, the term “sequence of interest” refers to a polynucleotide sequence, the use of which may be deemed desirable for a suitable purpose, in particular, for the manufacture of an mRNA for a therapeutic use, and includes but is not limited to, coding sequences of structural genes, and non-coding regulatory sequences that do not encode and mRNA or protein product.

As used herein, “in vitro transcription” or “IVT” refer to the process whereby transcription occurs in vitro in a non-cellular system to produce synthetic RNA molecules (e.g., synthetic mRNA) for use in various applications, including for therapeutic delivery to a subject, for example, as a therapeutic polynucleotide, which may be part of, or may be used to form, a therapeutic polynucleotide composition as described below. The therapeutic polynucleotide, (e.g., synthetic RNA molecules (transcription product)) generated may be combined with a delivery vehicle to form a therapeutic polynucleotide composition. Synthetic transcription products include mRNAs, antisense RNA molecules, shRNA, circular RNA molecules, ribozymes, and the like. An IVT reaction may use a purified linear DNA template comprising a promoter sequence and the sequence of the open reading frame (ORF) of a sequence of interest, ribonucleotide triphosphates or modified ribonucleotide triphosphates, a buffer system that includes DTT and magnesium ions, and a phage RNA polymerase.

As used herein a “therapeutic polynucleotide” refers to a polynucleotide (e.g., an mRNA) that may be part of a therapeutic polynucleotide composition for delivery to a subject to treat a symptom, disease, or condition in a subject; prevent a symptom, disease, or condition in a subject; or to improve or otherwise modify the subject's health.

As used herein a “therapeutic polynucleotide composition” (or “therapeutic composition” for short) may refer to a composition including one or more therapeutic polynucleotides (e.g., mRNA) encapsulated by a delivery vehicle, which composition may be administered to a subject in need thereof using any suitable administration routes, such as intratumoral, intramuscular, etc. injection. An example of a therapeutic polynucleotide composition is an mRNA (therapeutic) nanoparticle comprising at least one mRNA encapsulated by a delivery vehicle molecule. An mRNA vaccine is one example of a therapeutic polynucleotide composition.

As used herein, “delivery vehicle” refers to any substance that facilitates, at least in part, the in vivo, in vitro, or ex vivo delivery of a polynucleotide (e.g., therapeutic polynucleotide) to targeted cells or tissues (e.g., tumors, etc.). Referring to something as a delivery vehicle need not exclude the possibility of the delivery vehicle also having therapeutic effects. Some versions of a delivery vehicle may provide additional therapeutic effects. In some versions, a delivery vehicle may be a peptoid molecule, such as an amino-lipidated peptoid molecule, that may be used to at least partially encapsulate mRNA. The term “DV” will also be used herein as a shorthand for “delivery vehicle.”

As used herein, “joining” refers to methods such as ligation, synthesis, primer extension, annealing, recombination, or hybridization use to couple one component to another.

As used herein “purifying” refers to physical and/or chemical separation of a component (e.g., particles) of other unwanted components (e.g., contaminating substances, fragments, etc.).

As used herein, the term “substantially free” as used with respect to a given substance, includes 100% free of a given substance, or which comprises less than about 1.0%, or less than about 0.5%, or less than about 0.1% of the given substance.

### II. Overview of System Including Microfluidic Process Chip

FIG. 1 depicts examples of various components that may be incorporated into a system (100). System (100) of this example includes a housing (103) enclosing a seating mount (115) that may removably hold one or more microfluidic process chips (111). In other words, system (100) includes a chip-receiving component that is configured to removably accommodate a process chip (111), where the process chip (111) itself defines one or more microfluidic channels or fluid pathways. Components of system (100) (e.g., within housing (103)) that fluidically interact with process chip (111) may include fluid channels or pathways that are not necessarily considered microfluidic (e.g., with such fluid channels or pathways being larger than the microfluidic channels or fluid pathways in process chip (111)). In some versions, process chips (111) are provided and utilized as single-use devices, while the rest of system (100) is reusable. Housing (103) may be in the form of a chamber, enclosure, etc., with an opening that may be closed (e.g., via a lid or door, etc.) to thereby seal the interior. Housing (103) may enclose a thermal regulator and/or may be configured to be enclosed in a thermally-regulated environment (e.g., a refrigeration unit, etc.). Housing (103) may form an aseptic barrier. In some variations, housing (103) may form a humidified or humidity-controlled environment. In addition, or in the alternative, system (100) may be positioned in a cabinet (not shown). Such a cabinet may provide a temperature-regulated (e.g., refrigerated) environment. Such a cabinet may also provide air filtering and air flow management and may promote reagents being kept at a desired temperature through the manufacturing process. In addition, such a cabinet may be equipped with UV lamps for sterilization of process chip (111) and other components of system (100). Other suitable features may be incorporated into a cabinet that houses system (100).

In some scenarios, the assembly formed by housing (103) and the components of system (100) that are within housing (103), without process chip (111), may be considered as being an “instrument.” While controller (121) and user interface (123) are shown in FIG. 1 as being outside of housing (103), controller (121) and user interface (123) may in fact be provided in or on housing (103) and may thus also form part of the instrument. As described in greater detail below, this instrument may removably receive process chip (111) via a seating mount (115). When process chip (111) is seated in seating mount (115), the instrument and process chip (111) cooperate to together form system (100). When process chip (111) is removed from seating mount (115), the portion of system (100) that is left may be regarded as the “instrument.” The instrument, the system (100), and process chip (111) may each be considered an “apparatus.” The term “apparatus” may thus be read to include the instrument by itself, a process chip (111) by itself, the combination of the instrument and process chip (111), some other combination of components of system (100), or some other permutation of system (100) or components thereof.

Seating mount (115) may be configured to secure process chip (111) using one or more pins or other components configured to hold process chip (111) in a fixed and predefined orientation. Seating mount (115) may thus facilitate process chip (111) being held at an appropriate position and orientation in relation to other components of system (100). In the present example, seating mount (115) is configured to hold process chip (111) in a horizontal orientation, such that process chip (111) is parallel with the ground.

In some variations, a thermal control (113) may be located adjacent to seating mount (115), to modulate the temperature of any process chip (111) mounted in seating mount (115). Thermal control (113) may include a thermoelectric component (e.g., Peltier device, etc.) and/or one or more heat sinks for controlling the temperature of all or a portion of any process chip (111) mounted in seating mount (115). In some variations, more than one thermal control (113) may be included, such as to separately regulate the temperature of different ones of one or more regions of process chip (111). Thermal control (113) may include one or more thermal sensors (e.g., thermocouples, etc.) that may be used for feedback control of process chip (111) and/or thermal control (113).

As shown in FIG. 1, a fluid interface assembly (109) couples process chip (111) with a pressure source (117), thereby providing one or more paths for fluid (e.g., gas) at a positive or negative pressure to be communicated from pressure source (117) to one or more interior regions of process chip (111) as will be described in greater detail below. While only one pressure source (117) is shown, system (100) may include two or more pressure sources (117). In some scenarios, pressure may be generated by one or more sources other than pressure source (117). For instance, one or more vials or other fluid sources within reagent storage frame (107) may be pressurized. In addition, or in the alternative, reactions and/or other processes carried out on process chip (111) may generate additional fluid pressure. In the present example, fluid interface assembly (109) also couples process chip (111) with a reagent storage frame (107), thereby providing one or more paths for liquid reagents, etc., to be communicated from reagent storage frame (107) to one or more interior regions of process chip (111) as will be described in greater detail below.

In some versions, pressurized fluid (e.g., gas) from at least one pressure source (117) reaches fluid interface assembly (109) via reagent storage frame (107), such that reagent storage frame (107) includes one or more components interposed in the fluid path between pressure source (117) and fluid interface assembly (109). In some versions, one or more pressure sources (117) are directly coupled with fluid interface assembly, such that the positively pressurized fluid (e.g., positively pressurized gas) or negatively pressurized fluid (e.g., suction or other negatively pressurized gas) bypasses reagent storage frame (107) to reach fluid interface assembly (109). Regardless of whether the fluid interface assembly (109) is interposed in the fluid path between pressure source (117) and fluid interface assembly (109), fluid interface assembly (109) may be removably coupled to the rest of system (100), such that at least a portion of fluid interface assembly (109) may be removed for sterilization between uses. As described in greater detail below, pressure source (117) may selectively pressurize one or more chamber regions on process chip (111). In addition, or in the alternative, pressure source may also selectively pressurize one or more vials or other fluid storage containers held by reagent storage frame (107).

Reagent storage frame (107) is configured to contain a plurality of fluid sample holders, each of which may hold a fluid vial that is configured to hold a reagent (e.g., nucleotides, solvent, water, etc.) for delivery to process chip (111). In some versions, one or more fluid vials or other storage containers in reagent storage frame (107) may be configured to receive a product from the interior of the process chip (111). In addition, or in the alternative, a second process chip (111) may receive a product from the interior of a first process chip (111), such that one or more fluids are transferred from one process chip (111) to another process chip (111). In some such scenarios, the first process chip (111) may perform a first dedicated function (e.g., synthesis, etc.) while the second process chip (111) performs a second dedicated function (e.g., encapsulation, etc.). Reagent storage frame (107) of the present example includes a plurality of pressure lines and/or a manifold configured to divide one or more pressure sources (117) into a plurality of pressure lines that may be applied to process chip (111). Such pressure lines may be independently or collectively (in sub-combinations) controlled.

Fluid interface assembly (109) may include a plurality of fluid lines and/or pressure lines where each such line includes a biased (e.g., spring-loaded) holder or tip that individually and independently drives each fluid and/or pressure line to process chip (111) when process chip (111) is held in seating mount (115). Any associated tubing (e.g., the fluid lines and/or the pressure lines) may be part of fluid interface assembly (109) and/or may connect to fluid interface assembly (109). In some versions, each fluid line comprises a flexible tubing that connects between reagent storage frame (107), via a connector that couples the vial to the tubing in a locking engagement (e.g., ferrule) and process chip (111). In some versions, the ends of the fluid lines/pressure lines may be configured to seal against process chip (111) (e.g., at a corresponding sealing port formed in process chip (111)), as described below. In the present example, the connections between pressure source (117) and process chip (111), and the connections between vials in reagent storage frame (107) and process chip (111), all form sealed and closed paths that are isolated when process chip (111) is seated in seating mount (115). Such sealed, closed paths may provide protection against contamination when processing therapeutic polynucleotides.

The vials of reagent storage frame (107) may be pressurized (e.g., >1 atm pressure, such as 2 atm, 3 atm, 5 atm, or higher). In some versions, the vials may be pressurized by pressure source (117). Negative or positive pressure may thus be applied. For example, the fluid vials may be pressurized to between about 1 and about 20 psig (e.g., 5 psig, 10 psig, etc.). Alternatively, a vacuum (e.g., about −7 psig or about 7 psia) may be applied to draw fluids back into the vials (e.g., vials serving as storage depots) at the end of the process. The fluid vials may be driven at lower pressure than the pneumatic valves as described below, which may prevent or reduce leakage. In some variations, the difference in pressure between the fluid and pneumatic valves may be between about 1 psi and about 25 psi (e.g., about 3 psi, about 5 psi, 7 psi, 10 psi, 12 psi, 15 psi, 20 psi, etc.).

System (100) of the present example further includes a magnetic field applicator (119), which is configured to create a magnetic field at a region of the process chip (111). Magnetic field applicator (119) may include a movable head that is operable to move the magnetic field to thereby selectively isolate products that are adhered to magnetic capture beads within vials or other storage containers in reagent storage frame (107).

System (100) of the present example further includes one or more sensors (105). In some versions, such sensors (105) include one or more cameras and/or other kinds of optical sensors. Such sensors (105) may sense one or more of a barcode, a fluid level within a fluid vial held within reagent storage frame (107), fluidic movement within a process chip (111) that is mounted within seating mount (115), and/or other optically detectable conditions. In versions where a sensor (105) is used to sense barcodes, such barcodes may be included on vials of reagent storage frame (107), such that sensor (105) may be used to identify vials in reagent storage frame (107). In some versions, a single sensor (105) is positioned and configured to simultaneously view such barcodes on vials in reagent storage frame (107), fluid levels in vials in reagent storage frame (107), fluidic movement within a process chip (111) that is mounted within seating mount (115), and/or other optically detectable conditions. In some other versions, more than one sensor (105) is used to view such conditions. In some such versions, different sensors (105) may be positioned and configured to separately view corresponding optically detectable conditions, such that a sensor (105) may be dedicated to a particular corresponding optically detectable condition.

In versions where sensors (105) include at least one optical sensor, visual/optical markers may be used to estimate yield. For example, fluorescence may be used to detect process yield or residual material by tagging with fluorophores. In addition, or in the alternative, dynamic light scattering (DLS) may be used to measure particle size distributions within a portion of the process chip (111) (e.g., such as a mixing portion of process chip (111)). In some variations, sensor (105) may provide measurements using one or two optical fibers to convey light (e.g., laser light) into process chip (111); and detect an optical signal coming out of process chip (111). In versions where sensor (105) optically detects process yield or residual material, etc., sensor (105) may be configured to detect visible light, fluorescent light, an ultraviolet (UV) absorbance signal, an infrared (IR) absorbance signal, and/or any other suitable kind of optical feedback.

In versions where sensors (105) include at least one optical sensor that is configured to capture video images, such sensors (105) may record at least some activity on process chip (111). For example, an entire run for synthesizing and/or processing a material (e.g., a therapeutic RNA) may be recorded by one or more video sensors (105), including a video sensor (105) that may visualize process chip (111) (e.g., from above). Processing on process chip (111) may be visually tracked and this video record may be retained for later quality control and/or processing. Thus, the video record of the processing may be saved, stored, and/or transmitted for subsequent review and/or analysis. In addition, as will be described in greater detail below, the video may be used as a real-time feedback input that may affect processing using at least visually observable conditions captured in the video.

System (100) of the present example may be controlled by a controller (121). Controller (121) may include one or more processors, one or more memories, and various other suitable electrical components. In some versions, one or more components of controller (121) (e.g., one or more processors, etc.) is/are embedded within system (100) (e.g., contained within housing (103)). In addition, or in the alternative, one or more components of controller (121) (e.g., one or more processors, etc.) may be detachably attached or detachably connected with other components of system (100). Thus, at least a portion of controller (121) may be removable. Moreover, at least a portion of controller (121) may be remote from housing (103) in some versions.

The control by controller (121) may include activating pressure source (117) to apply pressure through process chip (111) to drive fluidic movement, among other tasks. Controller (121) may be completely or partially outside of housing (103); or completely or partially inside of housing (103). Controller (121) may be configured to receive user inputs via a user interface (123) of system (100); and provide outputs to users via user interface (123). In some versions, controller (121) is fully automated to a point where user inputs are not needed. In some such versions, user interface (123) may provide only outputs to users. User interface (123) may include a monitor, a touchscreen, a keyboard, and/or any other suitable features. Controller (121) may coordinate processing, including moving one or more fluid(s) onto and on process chip (111), mixing one or more fluids on process chip (111), adding one or more components to process chip (111), metering fluid in process chip (111), regulating the temperature of process chip (111), applying a magnetic field (e.g., when using magnetic beads), etc. Controller (121) may receive real-time feedback from sensors (105) and execute control algorithms in accordance with such feedback from sensors (105). Such feedback from sensors (105) may include, but need not be limited to, identification of reagents in vials in reagent storage frame (107), detected fluid levels in vials in reagent storage frame (107), detected movement of fluid in process chip (111), fluorescence of fluorophores in fluid in process chip (111), etc. Controller (121) may include software, firmware and/or hardware. Controller (121) may also communicate with a remote server, e.g., to track operation of the apparatus, to re-order materials (e.g., components such as nucleotides, process chips (111), etc.), and/or to download protocols, etc.

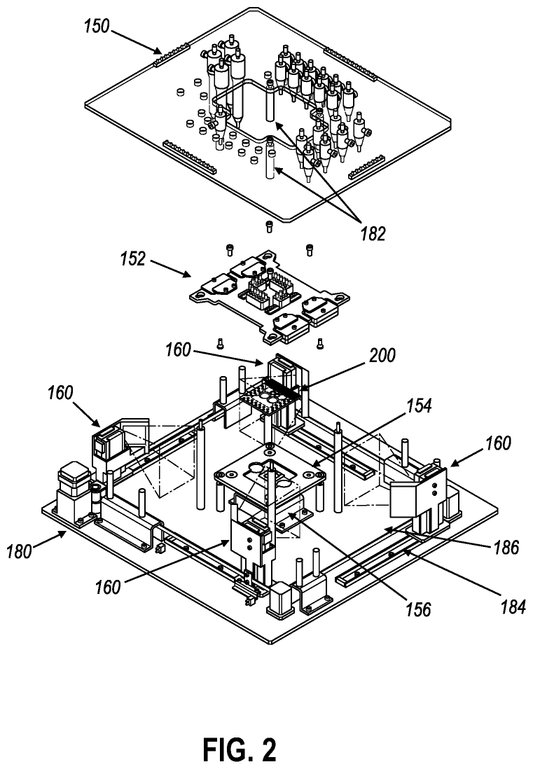

FIG. 2 shows examples of certain forms that may be taken by various components of system (100). In particular, FIG. 2 shows a reagent storage frame (150), a fluid interface assembly (152), a seating mount (154), a thermal control (156), and a process chip (200). Reagent storage frame (150), fluid interface assembly (152), seating mount (154), thermal control (156), and process chip (200) of this example may be configured and operable just like reagent storage frame (107), fluid interface assembly (109), seating mount (115), thermal control (113), and process chip (111), respectively, described above. These components are secured relative to a base (180). A set of rods (182) support reagent storage frame (150) over fluid interface assembly (152).

As shown in FIG. 2, a set of optical sensors (160) are positioned at four respective locations along base (180). Optical sensors (160) may be configured and operable like sensors (105) described above. Optical sensors (160) may include off-the-shelf cameras or any other suitable kinds of optical sensors. Optical sensors (160) are positioned such that fluid vials held within reagent storage frame (150) are within the field of view of one or more of optical sensors (160). In addition, process chip (200) is within the field of view of one or more of optical sensors (160). Each optical sensor (160) is movably secured to base (180) via a corresponding rail (184) (e.g., in a gantry arrangement), such that each optical sensor (160) is configured to translate laterally along each corresponding rail (184). A linear actuator (186) is secured to each optical sensor (160) and is thereby operable to drive lateral translation of each optical sensor (160) along the corresponding rail (184). Each actuator (186) may be in the form of a drive belt, a drive chain, a drive cable, or any other suitable kind of structure. Controller (121) may drive operation of actuators (186). Optical sensors (160) may be moved along rails (184) during operation of system (100) in order to facilitate viewing of the appropriate regions of vials in reagent storage frame (150) and/or process chip (200). In some scenarios, optical sensors (160) move in unison along corresponding rails (184). In some other scenarios, optical sensors (160) move independently along corresponding rails (184).

While optical sensors (160) are shown in FIG. 2 as being mounted to base (180), optical sensors (160) may be positioned elsewhere within system (100), in addition to or as an alternative to being mounted to base (180). For instance, some versions of reagent storage frame (107) may include one or more optical sensors (160) positioned and configured to provide an overhead field of view. In some such versions, such optical sensors (160) may be mounted to rails, movable cantilever arms, or other structures that allow such optical sensors (160) to be repositioned during operation of system (100). Optical sensors (160) may be positioned in any other suitable locations. While not shown, system (100) may also include one or more sources of light (e.g., electroluminescent panels, etc.) to provide illumination that aids in optical sensing by optical sensors (160).

In some versions, one or more mirrors are used to facilitate visualization of components of system (100) by optical sensors (160). Such mirrors may allow optical sensors (160) to view components of system (100) that may not otherwise be within the field of view of sensors (160). Such mirrors may be placed directly adjacent to optical sensors (160). In addition, or in the alternative, such mirrors may be placed adjacent to one or more components of system (100) that are to be viewed by optical sensors (160).

In use of system (100), an operator may select a protocol to run (e.g., from a library of preset protocols), or the user may enter a new protocol (or modify an existing protocol), via user interface (123). From the protocol, controller (121) may instruct the operator which kind of process chip (111) to use, what the contents of vials in reagent storage frame (107) should be, and where to place the vials in reagent storage frame (107). The operator may load process chip (111) into seating mount (115); and load the desired reagent vials and export vials into reagent storage frame (107). System (100) may confirm the presence of the desired peripherals, identify process chip (111), and scan identifiers (e.g., barcodes) for each reagent and product vial in reagent storage frame (107), facilitating the vials to match the bill-of-reagents for the selected protocol. After confirming the starting materials and equipment, controller (121) may execute the protocol. During execution, valves and pumps are actuated to deliver reagents as described in greater detail below, reagents are blended, temperature is controlled, and reactions occur, measurements are made, and products are pumped to destination vials in reagent storage frame (107).

### III. Example of Process Chip

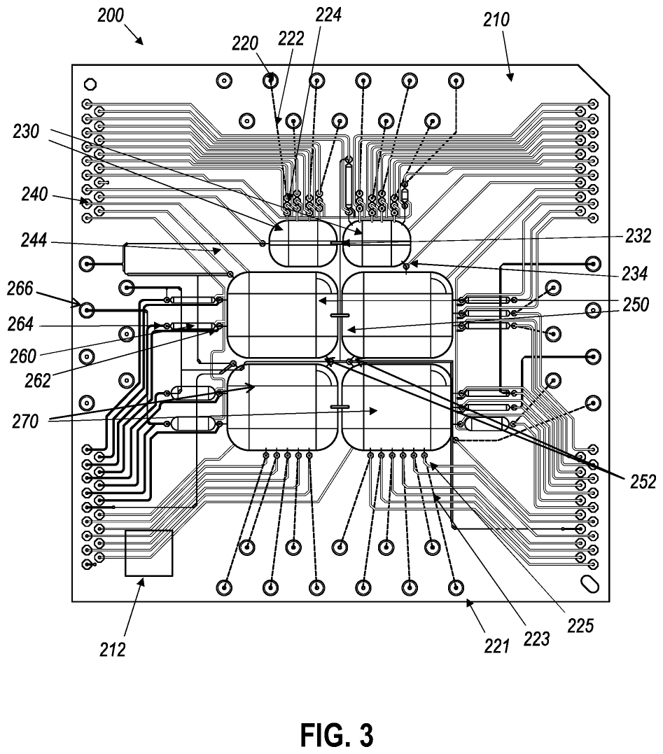

FIG. 3 depicts the example of a process chip (200) in further detail. In combination with the rest of system (100), process chip (200) may be utilized to provide in vitro synthesis, purification, concentration, formulation, and/or analysis of therapeutic compositions, including but not limited to therapeutic polynucleotides and therapeutic polynucleotide compositions. As shown in FIG. 3, process chip (200) of this example includes a plurality of fluid ports (220). Each fluid port (220) has an associated fluid channel (222) formed in process chip (200), such that fluid communicated into fluid port (220) will flow through the corresponding fluid channel (222). As described in greater detail below, each fluid port (220) is configured to receive fluid from a corresponding fluid line (206) from fluid interface assembly (109). In the present example, each fluid channel (222) leads to a valve chamber (224), which is operable to selectively prevent or permit fluid from the corresponding fluid channel (222) to be further communicated along process chip (200) as will be described in greater detail below.

As also shown in FIG. 3, process chip (200) of this example includes a plurality of additional chambers (230, 250, 270) that may be used to serve different purposes during the process of producing the therapeutic composition as described herein. By way of example only, such additional chambers (230, 250, 270) may be used to provide synthesis, purification, dialysis, compounding, and/or concentration of one or more therapeutic compositions; or to perform any other suitable function(s). Fluid may be communicated from one chamber (230) to another chamber (230) via a fluidic connector (232). In some versions, fluidic connector (232) is operable like a valve between an open and closed state (e.g., similar to valve chamber (224)). In some other versions, fluidic connector (232) remains open throughout the process of making the therapeutic composition. In the present example, chambers (230) are used to provide synthesis of polynucleotides, though chambers (230) may alternatively serve any other suitable purpose(s).

In the example shown in FIG. 3, another valve chamber (234) is interposed between one of chambers (230) and one of chambers (250), such that fluid may be selectively communicated from chamber (230) to chamber (250). Chambers (250) are provided in a pair and are coupled with each other such that process chip (200) may communicate the fluid back and forth between chambers (250). While a pair of chambers (250) are provided in the present example, any other suitable number of chambers (250) may be used, including just one chamber (250) or more than two chambers (250). Chambers (250) may be used to provide purification of the fluid and/or may serve any of the other various purposes described herein; and may have any suitable configuration. In versions where a chamber (250) is used for purification, chamber (250) may include a material that is configured to absorb selected moieties from a fluidic mixture in chamber (250). In some such versions, the material may include a cellulose material, which may selectively absorb double-stranded mRNA from a mixture. In some such versions, the cellulose material may be inserted in only one chamber (250) of a pair of chambers (250), such that upon mixing the fluid from the first chamber (250) of the pair to the second chamber (250), mRNA and/or some other component may be effectively removed from the fluidic mixture, which may then be transferred to another pair of chambers (270) further downstream for further processing or export. Alternatively, chambers (250) may be used for any other suitable purpose.

Additional valve chambers (252) are interposed between each chamber (250) and a corresponding chamber (270), such that fluid may be selectively communicated from chambers (250) to chambers (270) via valve chambers (252). Chambers (270) are also coupled with each other such that process chip (200) may communicate the fluid back and forth between chambers (270). Chambers (270) may be used to provide mixing of the fluid and/or may serve any of the other various purposes described herein; and may have any suitable configuration.

As shown in FIG. 3, chambers (270) are also coupled with additional fluid ports (221) via corresponding fluid channels (223) and valve chambers (225). Fluid ports (221), fluid channels (223), and valve chambers (225) may be configured an operable like fluid ports (220), fluid channels (222), and valve chambers (224) described above. In some versions, fluid ports (221) are used to communicate additional fluids to chambers (270). In addition, or in the alternative, fluid ports (221) may be used to communicate fluid from process chip (200) to another device. For instance, fluid from chambers (270) may be communicated via fluid ports (221) directly to another process chip (200), to one or more vials in reagent storage frame (107), or elsewhere.

Process chip (200) further includes several reservoir chambers (260). In this example, each reservoir chamber (260) is configured to receive and store fluid that is being communicated to or from a corresponding chamber (250, 270). Each reservoir chamber (260) has a corresponding inlet valve chamber (262) and outlet valve chamber (264). Each inlet valve chamber (262) is interposed between reservoir chamber (260) and the corresponding chamber (250, 270) and is thereby operable to permit or prevent the flow of fluid between reservoir chamber (260) and the corresponding chamber (250, 270). Each outlet valve chamber (264) is operable to meter the flow of fluid between reservoir chamber (260) and a corresponding fluid port (266). In some versions, each fluid port (266) is configured to communicate fluid from a corresponding vial in reagent storage frame (107) to a corresponding reservoir chamber (260). In addition, or in the alternative, each fluid port (266) may be configured to communicate fluid from a corresponding reservoir chamber (260) to a corresponding vial in reagent storage frame (107). In the present example, reservoir chambers (260) are used to provide metering of fluid communicated to and/or from process chip (200). Alternatively, reservoir chambers (260) may be utilized for any other suitable purposes, including but not limited to pressurizing fluid that is communicated to and/or from process chip (200).

As also shown in FIG. 3, process chip (200) of this example includes a plurality of pressure ports (240). Each pressure port (240) has an associated pressure channel (244) formed in process chip (200), such that pressurized gas communicated through pressure port (240) will be further communicated through the corresponding pressure channel (244). As described in greater detail below, each pressure port (240) is configured to receive pressurized gas from a corresponding pressure line (208) from fluid interface assembly (109). In the present example, each pressure channel (244) leads to a corresponding chamber (224, 225, 230, 234, 250, 252, 260, 262, 264, 270) to thereby provide valving or peristaltic pumping via such chambers (224, 225, 230, 234, 250, 252, 260, 262, 264, 270) as described in greater detail below.

Process chip (200) may also include electrical contacts, pins, pin sockets, capacitive coils, inductive coils, or other features that are configured to provide electrical communication with other components of system (100). In the example shown in FIG. 3, process chip (200) includes an electrically active region (212) includes such electrical communication features. Electrically active region (212) may further include electrical circuits and other electrical components. In some versions, electrically active region (212) may provide communication of power, data, etc. While electrically active region (212) is shown in one particular location on process chip, electrically active region (212) may alternatively be positioned at any other suitable location or locations. In some versions, electrically active region (212) is omitted.

### IV. Example of Method of Manufacture of Therapeutics

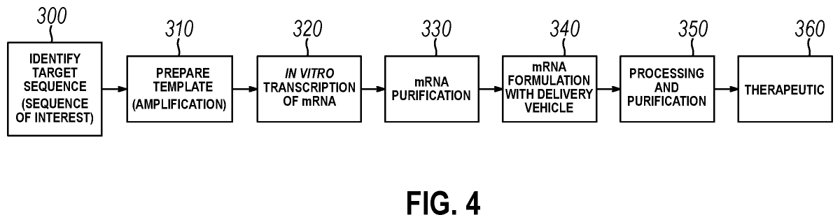

The above-described system may be used for the manufacture of mRNA-based therapeutics as described herein or other compositions. An example of a method for making an mRNA therapeutic is depicted in FIG. 4. In this example method, a target sequence (“sequence of interest”) is identified, as shown in block (300) of FIG. 4. A template comprising the target sequence (“sequence of interest”) may then be prepared and amplified (“amplification”), as shown in bock (310). Via in vitro transcription of mRNA as shown in block (320), mRNA is manufactured using a template comprising the target sequence. The resulting mRNA comprising the sequence of interest may then be purified, as shown in block (330), and then formulated with a DV, as shown in block (340). The resulting formulation comprising mRNA may then be further processed and optionally purified, as shown in block (360), for a therapeutic use, as shown in block (360). Examples of details of the method shown in FIG. 4 will be described further below.

Therapeutic uses of compositions yielded by the method shown in FIG. 4 may include, for example, cell therapies, oncological treatments, protein replacement, vaccines, expression of effector proteins, inducement of loss of function through expression of dominant negative proteins, and gene/genome editing. In addition to their high potency, mRNA therapeutics may also have benefits related to their rapid development cycle, standardized manufacturing, transient expression, and low risk of genomic integration. The methods and apparatuses described herein may be used to manufacture mRNA therapeutics for one or more of these categories of therapeutics.

### A. Identify Sequence of Interest

Any suitable method and criteria may be used to identify a sequence of interest for the part of the method represented by block (300) of FIG. 4. In some instances, the sequence of interest may be a short piece of DNA that encodes for a some or all of a product molecule (RNA or protein). The sequence of interest may be based, at least in part, on a specific patient's genetics (e.g., genotype), including generating a specific mRNA composition based on the patient's own sequence. The sequence of interest may additionally or alternatively be based, at least in part, on a specific patient's phenotype (e.g., based on the category a patient falls into, such as risk factor categories). In any case, through the system and method described herein, a composition may be compounded at the point-of-care to generate an optimized treatment for an individual.

### B. Prepare Template (Amplification)

Once the sequence of interest has been identified, a template containing the sequence of interest may be prepared and amplified, as shown in block (310). The template may be a DNA template, such as linear DNA, plasmid DNA, or combinations thereof. The template may comprise an in vitro transcription facilitator cassette (IFC). The IFC may be an in vitro transcription capable double-stranded DNA. The template may be incorporated into an IFC having functional elements that facilitate in vitro transcription (e.g., from an inserted sequence of interest), such as a promoter, a portion encoding a 5′ untranslated region, (5′UTR), a portion encoding a 3′ untranslated region (3′UTR), and a portion encoding for a polyA tail. The IFC may also include one or more linkers (e.g., at least one cleavable site) useful for cloning a sequence of interest into the in vitro transcription facilitator cassette for expression of the sequence of interest and restriction sites to allow for template linearization. An IFC may be manufactured synthetically or non-synthetically.

A sequence of interest useful for inserting into an IFC may be manufactured synthetically or non-synthetically. A sequence of interest may be cleaved prior to combining it with an IFC. In particular, a sequence of interest may be cleaved with the same restriction endonuclease(s) as used to cleave the IFC; but may also be generated through enzymatic amplification. In any case, a template generated in accordance with block (310) of the method shown in FIG. 4 may take various forms. In some versions, the template comprises a uracil-containing polynucleotide sequence.

### C. In Vitro Transcription

A template generated in accordance with block (310) of the method shown in FIG. 4 may be used for subsequent in vitro transcription (IVT) reactions to form a therapeutic polynucleotide, such as therapeutic mRNA, as shown in block (320) of FIG. 4. This IVT process may be conducted inside a process chip such as any of the process chips (111, 200) described herein, with the process being driven by controller (121). Part of this IVT process may include combining the template with reagents such as uracil-N-glycosylase (UNG) enzyme, dNTPs (including dUTP, modified dUTP, and combinations thereof), polymerase, and buffer. The IVT reaction may be incubated under controlled conditions to produce capped mRNA molecules. Following the IVT reaction, a DNAse treatment may be performed to degrade the template DNA. This may be performed inside the IVT reaction chamber, and parameters such as dilution rate, enzyme/buffer concentration, temperature and mixing may be controlled to optimized levels. This procedure may be executed autonomously and recorded by a monitoring camera (e.g., one or more of sensors (105)).

D. mRNA Purification

The mRNA generated through the through the IVT process may be purified, as shown in block (330), to remove impurities and side products. In some versions, this purification includes use of cellulose and an ethanol wash. For instance, a cellulose membrane may be used to selectively capture dsRNA under precisely controlled binding conditions and eluting the non-bound fraction a chamber of a process chip such as process chip (111, 200). Another purification step may use 1-2 um carboxyl-coated paramagnetic beads that selectively capture mRNA greater than 500 bp in length. One or more washes may be performed to remove unbound material, such as nucleotides, enzymes, and degraded template. Pure mRNA may then be eluted in USP grade water. A sampling chamber of a process chip (111, 200) may be used for analysis of the purified mRNA. The sampling chamber may receive detection reagents/probes for confirming the content of the resulting, purified mRNA.

E. mRNA Formulation With Delivery Vehicle

The purified mRNA may then be retrieved from the process chip (111, 200) for formulation with a DV, as shown in block (340). In some instances, this formulation process is carried out, at least in part, through a formulation version of process chip (111). Through the formulation process, the purified mRNA may be combined with at least one DV molecule to form an mRNA nanoparticle. For example, an aqueous solution of mRNA cargo may be combined with an ethanolic solution of DV in a microfluidic mixing structure within a formulation version of process chip (111). The material may then undergo two post-formulation processing steps involving first an on-chip dialysis process to exchange buffer components in the formulated product, followed by a concentration step to reduce the volume of the drug product to match specifications. The resulting formulation may yield encapsulated mRNA in the form of amphipathic nanoparticles (ANPs). In some versions, these ANPs are on the order of 100 nm in diameter, or smaller.

In some versions, the DV molecules may include lipitoid-based molecules, such as amino-lipidated peptoids. During the formulation process of block (340), the temperature of mixing stages on the formulation version of process chip (111) may be controlled to a temperature or range of temperatures (e.g., between about 2 degrees C. and about 20 degrees C.) that is calibrated to enhance mixing for mixing in the mixing stages. The enhanced mixing temperature may be based on the formulation being mixed (in some examples the sequence of the mRNA and/or the DV) within the particular geometry of the mixing chamber. Exposure of DV components to aqueous solution and interaction between cationic (+) lipids and anionic (−) mRNA may trigger particle formation. The mRNA may be dissolved in an acidic buffer, which may help ensure full protonation of basic functional groups (e.g., amines) on the DV responsible for its cationic charge. The DV may be dissolved in an aqueous-miscible organic solvent (e.g., ethanol) that facilitates the formation of nano-sized particles upon exposure to the aqueous cargo solution. Immediately after mixing, the solution pH may be stabilized by a neutral buffer.

In some versions, a peptoid-based lipid formulation may be used as the DV, which may incorporate both cationic groups and lipid moieties onto an N-substituted peptide (i.e., peptoid) backbone. The DV components may be monodisperse, fully-characterizable chemical entities. The DV may comprise one or more polyanionic compounds, one or more PEGylated (referring to covalent binding of polyethylene glycol (PEG) molecules) compounds, and one or more cationic compounds. Suitable cationic compounds may include but are not limited to cationic lipids, cationic lipid-peptide conjugates (e.g., lipitoids), cationic peptides, cationic polymers, and lipid-like (lipophilic) cationic compounds. The DV may comprise one or more tertiary amino lipidated and/or PEGylated cationic peptide compounds. The tertiary amino lipidated and/or PEGylated cationic peptide compounds may be peptide chains comprising N-substituted amino acid residues.

A formulation version of process chip (111) may control, with precision, the mixing rate of the material. Faster or slower mixing may be provided and controlled by controller (121). In some versions, immediately following mixing, the ANPs may be diluted with an in-line addition of 1:1 neutral PBS. This may neutralize an acidic formulation buffer and may prepare the formulation for dialysis and concentration. The ANPs created through the formulation process of block (340) may also be evaluated on the formulation version of process chip (111). For instance, the formulation process of block (340) may include a one or more dynamic light scattering (DLS) stages to evaluate particle size, particle distribution, and/or other characteristics of the ANPs. In addition, or in the alternative, a fluorescent mRNA-specific probe may be used to determine RNA concentration before and after particle disruption by addition of a detergent. This assay may elucidate the mRNA concentration for dosing information and the percentage of mRNA encapsulated in the ANPs versus free in solution. Other methods may be used.

### F. Post-Formulation

Once ANPs are formed during the formulation process of block (340), several post-processing operations may be completed on the formulation version of process chip (111), as shown in block (350) of FIG. 4. In some versions, these additional processes may include dialysis for buffer exchange and ethanol removal, followed by evaporative concentration to reduce volume for dosing. Other suitable processing steps may be used. Ultimately, the process may yield a ready-to-use therapeutic polynucleotide composition, as shown in block (360). Such therapeutic compositions may include, but are not limited to, cell therapies, oncological treatments, protein replacement, vaccines, expression of effector proteins, inducement of loss of function through expression of dominant negative proteins, and gene/genome editing. Such therapeutic compositions may be delivered to patients in any suitable fashion.

The various sub-processes referred to in FIG. 4 may be carried out using any suitable number or type(s) of process chip (111). In some versions, the entire process shown in FIG. 4 is carried out using a single version of process chip (111). In some other versions, certain sub-processes are carried out on a dedicated process chip (111), while other sub-processes are carried out on another dedicated process chip (111). For instance, in some versions, template preparation (block 310) is carried out on a dedicated template version of process chip (111); IVT transcription and purification (blocks 320, 330) are carried out on a dedicated IVT version of process chip (111); and formulation (block 340) is carried out on a dedicated formulation version of process chip (111).

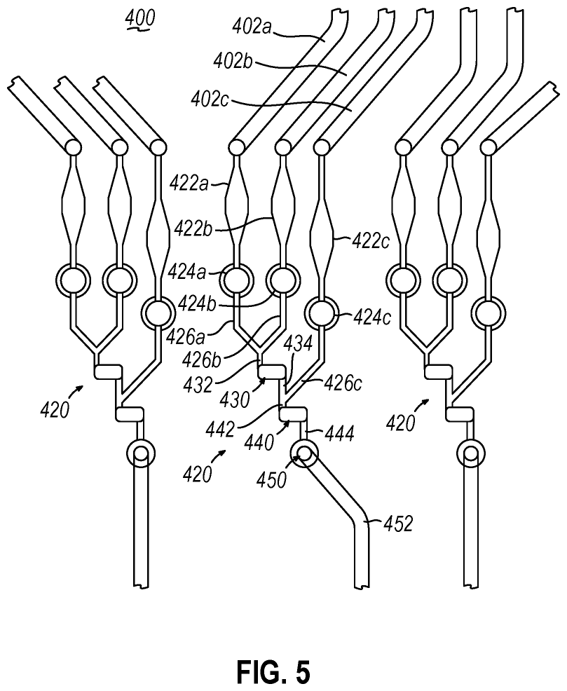

FIG. 5 shows a portion of a process chip (400) that has features that may be used to carry out at least some of the formulation process (block 340). Process chip (400) of this example includes a plurality of fluid channels (402). Each fluid channel (402) has a fluid port (not shown), such that fluid may be communicated to fluid channels (402) via corresponding fluid ports. Some of these fluid ports may receive fluid from corresponding vials in reagent storage frame (107). In addition, or in the alternative, some of these fluid ports may receive fluid from corresponding fluid outputs of another process chip (111, 200). Alternatively, the fluid ports leading to fluid channels (402) may receive fluid from any other suitable sources.

Fluid channels (402) lead to several mixing assemblies (420) that are integrated into process chip (400). In some versions, all mixing assemblies (420) on a process chip (400) have the same kinds of fluid inputs and are intended to all generate the same kind of fluid output. Each mixing assembly (420) includes a set of vacuum caps (422), a set of inlet valves (424), and a set of mixing chambers (430, 440). Referring to one mixing assembly (420) as being representative of the other mixing assemblies (420), mixing assembly (420) includes a first vacuum cap (422a), which receives fluid from a first fluid channel (402a); a second vacuum cap (422b), which receives fluid from a second fluid channel (402b); and a third vacuum cap (422c), which receives fluid from a third fluid channel (402c). Each vacuum cap (422a, 422b, 422c) is configured to evacuate air or other gas from the corresponding fluid channel (402a, 402b, 402c), such that vacuum caps (422a, 422b, 422c) may clear any bubbles, etc., that might otherwise be present. A first valve (424a) selectively prevents or permits the flow of fluid from first vacuum cap (422a) into a first inlet channel (426a) leading toward first mixing chamber (430). A second valve (424b) selectively prevents or permits the flow of fluid from second vacuum cap (422b) into an inlet channel (426b) leading toward first mixing chamber (430). Channels (426a, 426b) converge to form an inlet channel (432) leading into first mixing chamber (430). The fluids from channels (426a, 426b) are thus mixed together within first mixing chamber (430).

A third valve (424c) selectively prevents or permits the flow of fluid from third vacuum cap (422c) into a third channel (426c) leading toward second mixing chamber (440). An outlet channel (434) from first mixing chamber (430) converges with third channel (426c) to form an inlet channel (442) leading into second mixing chamber (440). The fluids from channels (434, 426c) are thus mixed together within second mixing chamber (440). The fluid mixed in second mixing chamber (440) is output through an outlet channel (444).

In some versions where process chip (400) is used to provide encapsulated mRNA, a combination of mRNA and a formulation buffer may be communicated through first fluid channel (402a) and a DV molecule or molecules in ethanol may be communicated through second fluid channel (402b). In some versions, the formulation buffer includes an aqueous buffer such as a phosphate-citrate buffer solution at a slightly acidic condition (e.g., having a pH of approximately 6.0). Alternatively, any other suitable formulation buffer may be used. The mRNA and DV molecules may thus be combined for encapsulation in first mixing chamber (430). A dilution agent (e.g., a phosphate buffer saline (PBS) solution, etc.) may be communicated through third fluid channel (402c). In such versions, second mixing chamber (440) may thus be used to provide pH adjustment. In some variations, the mRNA and formulation buffer are combined in another mixing chamber (not shown) that is upstream of first fluid channel (402a). Similarly, the DV molecules and ethanol may be combined in another mixing chamber (not shown) that is upstream of second fluid channel (402b).

An additional channel (452) is fluidically coupled with outlet channel (444) via an opening (450). Channel (452) may be fluidically coupled with a collection vial in reagent storage frame (107) (e.g., for storage, etc.), with another process chip (111, 200) (e.g., for further processing, etc.), or with anything else.

In versions where certain sub-processes are carried out on a dedicated process chip (111) while other sub-processes are carried out on another dedicated process chip (111), the same instrument of system (100) may be used with the various process chips (111). In some such versions, the same instrument of system (100) accommodates all the process chips (111) that are needed to carry out the process shown in FIG. 4, such that the instrument of system (100) transfers fluids from one process chip (111) to another process chip (111) at the appropriate stage of the process. In some other versions, an instrument of system (100) only accommodates one single process chip (111) at a time. In some such versions, a portion of the process of FIG. 4 (e.g., template preparation (block (310)) may be carried out using a dedicated process chip (111), with the resulting fluid(s) being stored in one or more vials in reagent storage frame (107). That dedicated process chip (111) may then be removed from the instrument of system (100) and be replaced with another dedicated process chip (e.g., a version of process chip (111) dedicated to performing IVT transcription (block (320))), with that second dedicated process chip receiving fluid from one or more vials in reagent storage frame (107) and/or other sources. Different dedicated process chips (111) may thus be used in an appropriate sequence within the instrument of system (100) to carry out the process of FIG. 4. In some variations, different instruments of system (100) may be used with different respective dedicated process chips (111); instead of the same single instrument being used with different dedicated process chips (111).

### V. Example of Automated Fluid Delivery System

In some scenarios, it may be desirable to provide additional fluid processing capabilities to a system like system (100). For instance, it may be desirable to provide an adjunct fluid processing assembly that interfaces with components of system (100) to allow a user to readily test several samples of reagents in a process carried out through system (100); and readily retrieve several samples of compositions generated through system (100) using the samples of reagents. In some such scenarios, a user may wish to test several samples of mRNA fluid (e.g., a combination of mRNA and formulation buffer, as generated prepared through the IVT and purification processes described above with reference to blocks (320, 330) of FIG. 4) and several samples of DV fluid (e.g., DV molecules in ethanol, as described above) in a formulation process carried out in a process chip (e.g., like process chip (400)), in a formulation process described above with reference to block (340) of FIG. 4); and retrieve the several samples of encapsulated mRNA compositions (e.g., in the form of ANPs) that are formed during the formulation process.

While experimental testing such as that described above may be carried out in system (100), such as by preloading reagent storage frame (107) with the several reagent samples and retrieving the samples of encapsulated mRNA compositions from reagent storage frame (107), an adjunct fluid processing assembly may allow the user to more easily provide a large number of discrete reagent samples and collect a large number of discrete encapsulated mRNA composition samples (e.g., 96 discrete encapsulated mRNA composition samples). In other words, reagent storage frame (107) may, by itself, only have a capacity to hold a certain number of reagent samples, which may limit the usability of reagent storage frame (107) to screen a large number of conditions (e.g., different reagents). For instance, some versions of reagent storage frame (107) may involve a user switching vials in reagent storage frame (107), wash fluid communication channels leading to a process chip and within a process chip, and/or perform other potentially time-consuming operations. An adjunct fluid processing assembly may provide additional fluid storage and processing capabilities relative to the capabilities of reagent storage frame (107), thereby enhancing the number of conditions that may be screened, automating the use of different reagent samples, and automating the washing of fluid channels between reagent samples. An adjunct fluid processing assembly may also provide precise extraction of reagents to thereby prevent or otherwise reduce waste. Several examples of how an adjunct fluid processing assembly may be combined with, or incorporated into, variations of system (100) will be described in greater detail below.

### A. Example of Arrangements of Adjunct Fluid Processing Assemblies

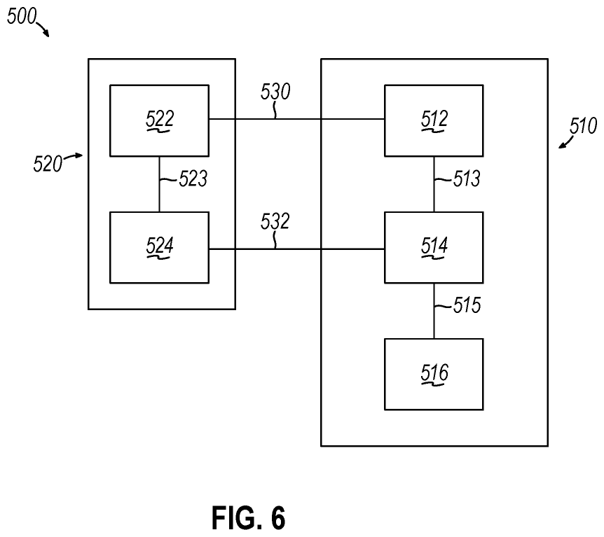

FIG. 6 shows an example of a system (500) that includes an instrument (510) and a separate fluid processing subsystem (520). Instrument (510) of this example may be configured and operable like the instrument of system (100). For instance, instrument (510) of this example includes a controller (512) and an integral fluid processing assembly (514). Controller (512) may be configured and operable like controller (121). Fluid processing assembly (514) may be configured and operable like the combination of pressure source (117), reagent storage frame (107), and fluid interface assembly (109). Controller (512) is coupled with fluid processing assembly (514) via an electrical communication pathway (513), which may include a plurality of wires, traces, other conductive paths, wireless couplings, etc. Controller (512) is thus operable to drive operation of fluid processing assembly (514) via electrical communication pathway (513).

Instrument (510) of this example is also operable to removably receive a process chip (516), which may be configured and operable like any of the variations of process chip (111) described herein. Fluid processing assembly (514) may be coupled with process chip (516) via a fluid communication pathway (515), which may include a plurality of tubes, other fluid conduits, etc. Instrument (510) may also have other components and functionalities similar to those described above with respect to the instrument of system (100).

Fluid processing subsystem (520) of this example includes a controller (522) and a fluid processing assembly (524). Controller (522) may be configured and operable like other controllers (121, 512) described herein. Controller (522) is coupled with fluid processing assembly (524) via an electrical communication pathway (523), which may include a plurality of wires, traces, other conductive paths, wireless couplings, etc. Controller (522) is thus operable to drive operation of fluid processing assembly (524) via electrical communication pathway (523). Controller (522) of fluid processing subsystem (520) is also coupled with controller (512) of instrument (510) via an electrical communication pathway (530), which may include a plurality of wires, traces, other conductive paths, wireless couplings, etc. In some versions, controller (522) may communicate commands, data, and/or other signals to controller (512) via electrical communication pathway (530). In addition, or in the alternative, controller (512) may communicate commands, data, and/or other signals to controller (522) via electrical communication pathway (530). In some variations, electrical communication pathway (530) is omitted, such that controllers (512, 522) are not in electrical communication with each other.

Fluid processing assembly (524) of fluid processing subsystem (520) is coupled with fluid processing assembly (514) of instrument (510) via a fluid communication pathway (532), which may include a plurality of tubes, other conduits, etc. In some versions, fluid processing assembly (524) may communicate fluids to fluid processing assembly (514) via fluid communication pathway (532). In addition, or in the alternative, fluid processing assembly (514) may communicate fluids to fluid processing assembly (524) via fluid communication pathway (532).

Fluid communication pathway (532) may be configured such that fluid communication pathway (532) may be readily separated from, and reconnected with, one or both of fluid processing assemblies (514, 524). Similarly, in versions where electrical communication pathway (530) is present, electrical communication pathway (530) may be configured such that electrical communication pathway (530) may be readily separated from, and reconnected with, one or both of controllers (512, 522). Thus, in some versions, fluid processing subsystem (520) may be readily separated from, and reconnected with, instrument (510). This may be desirable to accommodate different kinds of uses of instrument (510). For instance, some uses of instrument (510) may warrant the additional fluid processing functionality provided via fluid processing subsystem (520), as will be described in greater detail below, in which case a user may wish to couple fluid processing subsystem (520) with instrument (510). Other uses of instrument (510) may not warrant the additional fluid processing functionality provided via fluid processing subsystem (520); in which case a user may wish to decouple fluid processing subsystem (520) from instrument (510).

As an example of how fluid processing assemblies (514, 524) may be used together, a set of reagents may be transferred from fluid processing assembly (524) to process chip (516) via fluid processing assembly (514) and fluid communication pathways (515, 532). These reagents may be processed together via process chip (516) to form a composition. In some such scenarios, one or more other reagents residing on fluid processing assembly (514) (e.g., in a vial supported by a structure like reagent storage frame (107)) may be combined with one or more reagents from fluid processing assembly (524) on process chip (516). The resulting composition may ultimately be communicated back to fluid processing assembly (524) via fluid processing assembly (514) and fluid communication pathways (515, 532). The composition may then be retrieved from fluid processing assembly (524) for further processing. Alternatively, fluid processing assemblies (514, 524) may be used together in any other suitable fashion.

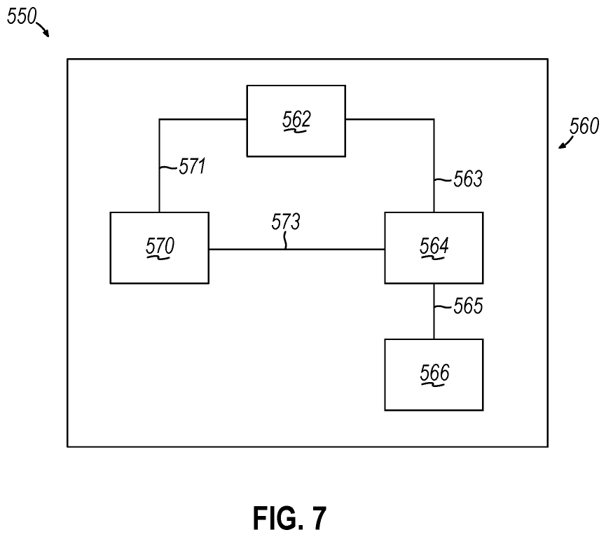

In system (500) of FIG. 6, instrument (510) and fluid processing subsystem (520) are provided as separate components that may be removably coupled together. In some scenarios, it may be desirable to integrate all the features and functionalities of instrument (510) and fluid processing subsystem (520) into a single instrument. To that end, FIG. 7 shows an example of a system (550) that includes a single instrument (560) that removably receives a process chip (566), which may be configured and operable like any of the variations of process chip (111) described herein. Instrument (560) of this example includes a controller (562), a first fluid processing assembly (564), and a second fluid processing assembly (570). Controller (562) may be configured and operable like other controllers (121, 512, 522) described herein. Controller (562) is coupled with first fluid processing assembly (564) via a first electrical communication pathway (563); and with second fluid processing assembly (570) via a second electrical communication pathway (571). Each electrical communication pathway (563, 571) may include a plurality of wires, traces, other conductive paths, wireless couplings, etc. Controller (562) is thus operable to drive operation of first fluid processing assembly (564) via first electrical communication pathway (563); and operation of second fluid processing assembly (570) via second electrical communication pathway (570). While both fluid processing assemblies (564, 570) share the same controller (562) in this example, other versions may provide separate controllers for fluid processing assemblies (564, 570), with such separate controllers being in communication with each other.

First fluid processing assembly (564) may be configured and operable like fluid processing assembly (514) described above. Second fluid processing assembly (570) may be configured and operable like fluid processing assembly (524). First fluid processing assembly (564) is coupled with second fluid processing assembly (570) via a fluid communication pathway (573), which may include a plurality of tubes, other conduits, etc. First fluid processing assembly (564) may also be coupled with process chip (566) via a fluid communication pathway (565), which may include a plurality of tubes, other fluid conduits, etc. In view of the foregoing, system (550) may be operated like system (500). While second fluid processing assembly (570) is integrated into instrument (560) instead of being part of a separate subassembly in this example, some versions of system (550) may nevertheless permit second fluid processing assembly (570) to be selectively coupled with, and decoupled from, controller (562) and first fluid processing assembly (564). In such versions, the presence of second fluid processing assembly (570) may be chosen by the user based on the intended use of system (550).

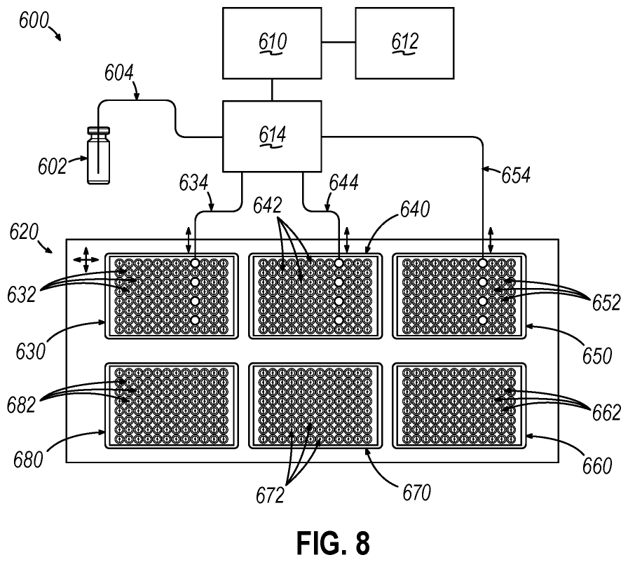

FIG. 8 shows an example of a system (600) that may represent a variation of system (500) and/or system (600) in the context of an illustrative use. In this example, system (600) includes a first fluid processing assembly (610), a process chip (612), and a second fluid processing assembly (614). First fluid processing assembly (610) may be configured and operable like fluid processing assemblies (514, 564). Process chip (612) may be configured and operable like any of the variations of process chip (111, 200) described herein. Second fluid processing assembly (614) may be configured and operable like fluid processing assemblies (524, 570). Fluid processing assemblies (610, 614) may be integrated into a single instrument (e.g., similar to system (550)); or provided separately and coupled together (e.g., similar to system (500)).

System (600) of this example further includes a tray support platform (620), with a plurality of trays (630, 640, 650, 660, 670, 680) arranged in a grid on an upper surface (622) of platform (620). Each tray (630, 640, 650, 660, 670, 680) defines a plurality of wells (632, 642, 652, 662, 672, 682). Each well (632, 642, 652, 662, 672, 682) is configured to hold a volume of fluid. Fluid processing assembly (614) includes a plurality of fluid communication pathways (634, 644, 654) that are configured to provide communication of fluid from and/or to wells (632, 642, 652, 662, 672, 682). As will be described in greater detail below, fluid processing assembly (614) may be operated such that fluid communication pathways (634, 644, 654) move in relation to trays (630, 640, 650, 660, 670, 680) to selectively communicate with wells (632, 642, 652, 662, 672, 682). As will also be described in greater detail below, tray support platform (620) may also move in relation to fluid communication pathways (634, 644, 654) to enable fluid communication pathways (634, 644, 654) to reach different wells (632, 642, 652, 662, 672, 682).

As also shown in FIG. 8, a separate vial (602) may be coupled with fluid processing assembly (614) via a fluid communication pathway (604). Fluid communication pathway (604) is configured to provide a path for fluid communication from vial (604) to fluid processing assembly (614). As shown, vial (602) is separate from tray support platform (620) in this example. In some versions, vial (602) is integrated into an instrument that contains fluid processing assembly (610) and process chip (612). For instance, vial (602) may be integrated into an assembly like reagent storage frame (107). While only one vial (602) is shown, system (600) may include more than one vial (602). Moreover, a vial (602) is just one example of a fluid-containing structure that may be provided separately from tray support platform (6200. Other suitable kinds of fluid-containing structures may be used.

In an example of use for system (600), system (600) may be used to perform mRNA formulation as described above in the context of block (340) of FIG. 4. In some such scenarios, process chip (612) of system (600) may be configured and operated like process chip (400) shown in FIG. 5. Each tray (630, 640, 650, 660, 670, 680) may be dedicated to serve a certain purpose. For instance, tray (630) may serve as a collection tray, such that wells (632) receive encapsulated mRNA that was formulated on process chip (612). Such encapsulated mRNA (e.g., in the form of ANPs) may be communicated to wells (632) of tray (630) via fluid processing assemblies (610, 614) and fluid communication pathway (634). When process chip (612) is configured like process chip (400), the encapsulated mRNA may be communicated from channels like channel (452). As will be described in greater detail below, fluid processing assemblies (610, 614) and fluid communication pathway (634) may communicate encapsulated mRNA from several channels like channel (452) in process chip (612) to several corresponding wells (632) of tray (630) simultaneously.

Tray (640) may serve as an mRNA source tray, such that wells (642) contain mRNA fluid that is used in the formulation process on process chip (612). Such mRNA fluid may include a combination of mRNA and formulation buffer; and may be prepared through the IVT and purification processes described above with reference to blocks (320, 330) of FIG. 4. Such mRNA fluid from tray (640) may be communicated to process chip (612) via fluid processing assemblies (610, 614) and via fluid communication pathway (644). When process chip (612) is configured like process chip (400), the mRNA fluid from tray (640) may be communicated to channels like channel (402a). As will be described in greater detail below, fluid processing assemblies (610, 614) and fluid communication pathway (644) may communicate mRNA fluid from several wells (642) to several corresponding channels like channel (402a) on process chip (612) simultaneously.

Tray (650) may serve as a DV fluid source tray, such that wells (642) contain DV fluid that is used in the formulation process on process chip (612). Such DV fluid may include DV molecules in ethanol, as described above. Such DV fluid from tray (650) may be communicated to process chip (612) via fluid processing assemblies (610, 614) and via fluid communication pathway (654). When process chip (612) is configured like process chip (400), the DV fluid from tray (640) may be communicated to channels like channel (402b). As will be described in greater detail below, fluid processing assemblies (610, 614) and fluid communication pathway (654) may communicate DV fluid from several wells (652) to several corresponding channels like channel (402b) on process chip (612) simultaneously.

Trays (660, 670) may serve as rinse fluid source trays, such that wells (662, 670) contain rinse fluid that is used to rinse fluid communication pathways (644, 654). When process chip (612) is configured like process chip (400), the rinse fluid may also rinse channels (402a, 402b) and structures downstream of channels. Such rinse fluid may include a combination of water and ethanol. Alternatively, any other suitable rinse fluid may be used. In some versions, rinse fluid in tray (660) is used to rinse components of fluid communication pathway (654) and channel (402b) while rinse fluid in tray (670) is used to rinse components of fluid communication pathway (644) and channel (402a). In some other versions, rinse fluid in tray (660) is used to perform a first rinsing stage for components of fluid communication pathways (644, 654); while rinse fluid in tray (670) is used to perform a second rinsing stage for components of fluid communication pathways (644, 654). In some such versions, the rinse fluid in tray (660) is different from the rinse fluid in tray (670).

Trays (680) may be used to collect waste from the rinsing process referred to above. Wells (682) may thus receive waste fluid from fluid communication pathway (634). When process chip (612) is configured like process chip (400), wells (682) may also receive waste from channel (452) and structures upstream of channel (452).

Vial (602) may provide a dilution agent (e.g., a PBS solution, etc.) that is used in the formulation process on process chip (612). Such buffer solution from vial (602) may be communicated to process chip (612) via fluid processing assemblies (610, 614) and via fluid communication pathway (604). When process chip (612) is configured like process chip (400), the dilution agent from vial (602) may be communicated to channels like channel (402c). As will be described in greater detail below, fluid processing assemblies (610, 614) and fluid communication pathway (604) may communicate dilution agent from vial (602) to several corresponding channels like channel (402c) on process chip (612) simultaneously.

In some other variations, vial (602) may contain mRNA that is used in the formulation process, while wells (642) may contain the buffer solution. As another variation, vial (602) may contain the DV fluid, while wells (652) may contain the buffer solution. As yet another variation, the buffer solution, mRNA, and DV fluid may all be contained in their own respective trays, such that vial (602) may be omitted.

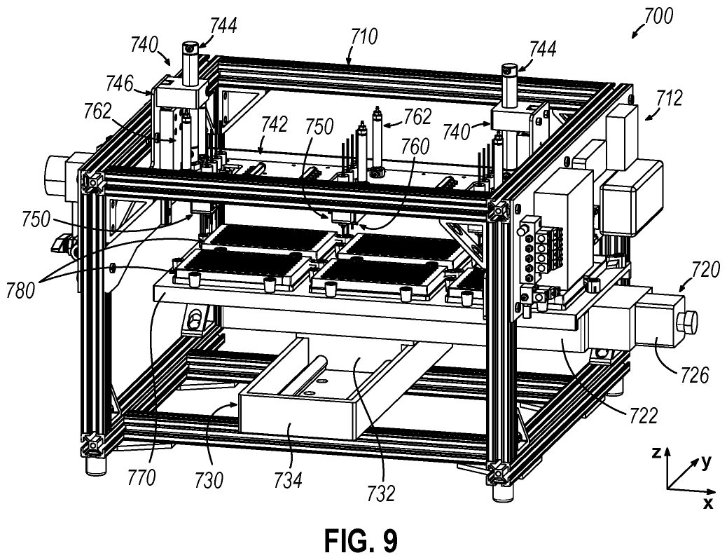

FIGS. 9-11B show an example of an adjunct fluid processing assembly (700) that may be combined with, or otherwise incorporated into, a variation of system (100). For instance, fluid processing assembly (700) of FIG. 9 may represent a from that may be taken by fluid processing subsystem (520) of FIG. 6. Alternatively, fluid processing assembly (700) of FIG. 9 may represent a from that may be taken by fluid processing assembly (570) of FIG. 7. Alternatively, fluid processing assembly (700) of FIG. 9 may represent a from that may be taken by fluid processing assembly (614) of FIG. 8. Alternatively, fluid processing assembly (700) of FIG. 9 may be combined with, or otherwise incorporated into, a variation of system (100) in any other suitable fashion.

Fluid processing assembly (700) of this example includes a frame (710) that structurally supports the rest of the components of fluid processing assembly (700). Fluid processing assembly (700) further includes a control circuit assembly (712) that is secured to a side of frame (710) and that includes several electrical components that are operable to drive the electrically-powered components of fluid processing assembly (700). Control circuit assembly (712) may be considered as being analogous to controller (522) or at least a portion of controller (562). Fluid processing assembly (700) further includes a pneumatic assembly (714) secured to another side of frame (710). Pneumatic assembly (714) includes one or more pumps, flow regulators, manifolds, and/or other components that are used to provide pressurized air for driving pneumatic components of fluid processing assembly (700) as described in greater detail below.

Fluid processing assembly (700) further includes a first tray drive assembly (720), a second tray drive assembly (730), a head support actuation assembly (740), a plurality of sampling head assemblies (750), a plurality of tray engagement assemblies (760), and a tray support platform (770). Tray support platform (770) is configured to removably receive and support several trays (780). As will be described in greater detail below, tray drive assemblies (720, 730) are operable to drive tray support platform (770) relative to frame (710) along two dimensions on a horizontal plane to thereby selectively position trays (780) relative to sampling head assemblies (750). As will also be described in greater detail below, head support actuation assembly (740) is operable to drive sampling head assemblies (750) along a vertical dimension to thereby selectively raise and lower sampling head assemblies (750) relative to trays (780). As will also be described in greater detail below, tray engagement assemblies (760) are operable to selectively engage trays (780) during at least part of the vertical range of travel of sampling head assemblies (750) relative to trays (780). The foregoing list of components and functionalities of fluid processing assembly (700) are not intended to be exhaustive. Other components and functionalities may be incorporated into fluid processing assembly (700).

### B. Examples of Features to Selectively Address Trays

As noted above, and as shown in FIGS. 9-11B, fluid processing assembly (700) includes a first tray drive assembly (720) and a second tray drive assembly (730), which are operable to drive tray support platform (770) relative to frame (710) along two dimensions on a horizontal plane. First tray drive assembly (720) includes a base (722) and a carriage (not shown). The carriage is positioned and configured to support tray support platform (770) and slide longitudinally along base (722). First tray drive assembly (720) further includes a motor (726) that is operable to drive motion of the carriage along base (722), and thereby drive tray support platform (770) longitudinally along base (722). Motor (726) may be coupled with control circuit assembly (712) via one or more cables, etc., such that control circuit assembly (712) may control activation of motor (736). In some versions, first tray drive assembly (720) further includes a screw gear that is coupled with motor (726); and that is further coupled with a nut feature of the carriage such that the screw gear and nut convert rotary motion from motor (726) into longitudinal motion of the carriage and tray support platform (770). Alternatively, any other suitable components may be used.

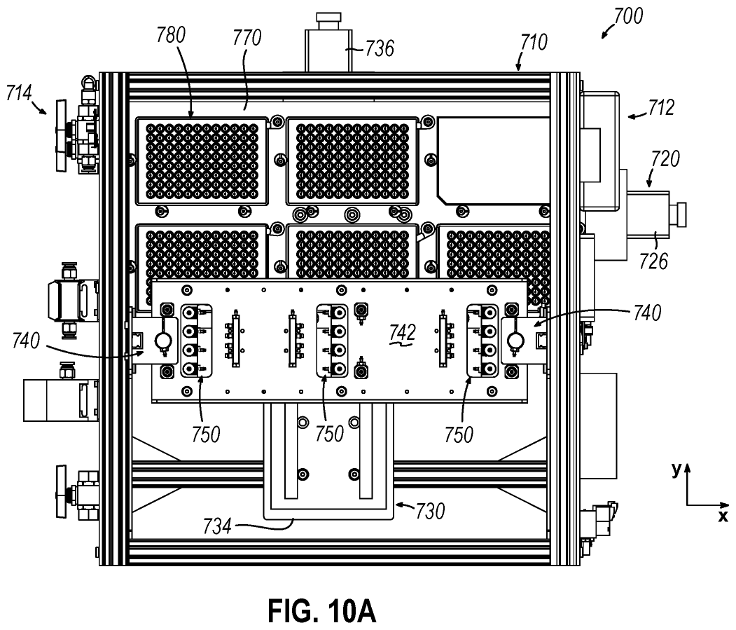

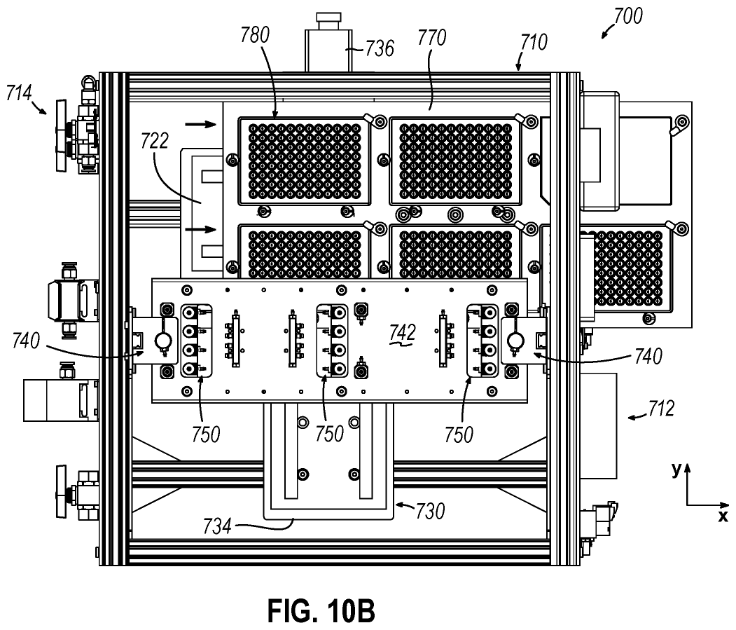

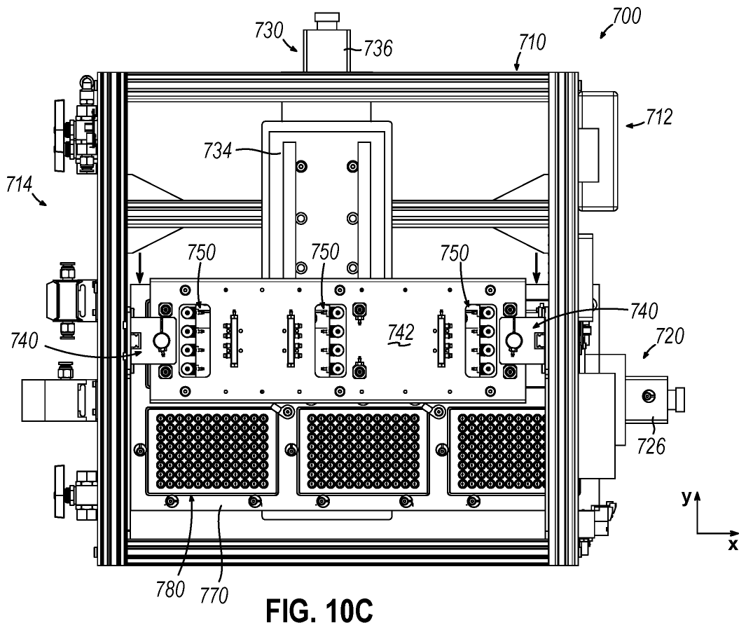

FIGS. 10A and 10B show an example of how first tray drive assembly (730) may drive tray support platform (770) along a horizontal “x” dimension. FIG. 10A shows tray support platform (770) at a first position along the “x” dimension while FIG. 10B shows tray support platform (770) at a second position along the “x” dimension. Such selective positioning of tray support platform (770) along the “x” dimension may enable certain wells (782) of trays (780) to be selectively positioned in relation to sampling head assemblies (750), as will be described in greater detail below.

Second tray drive assembly (730) includes a base (734) and a carriage (732). Carriage (734) is positioned and configured to support first tray drive assembly (720) and slide longitudinally along base (734). Second tray drive assembly (730) further includes a motor (736) that is operable to drive motion of the carriage along base (734), and thereby drive first tray drive assembly (720) longitudinally along base (734). Motor (736) may be coupled with control circuit assembly (712) via one or more cables, etc., such that control circuit assembly (712) may control activation of motor (736). As first tray drive assembly (720) is driven longitudinally along base (734), tray support platform (770) will be driven longitudinally with first tray drive assembly (720) along base (734). In some versions, second tray drive assembly (730) further includes a screw gear that is coupled with motor (736); and that is further coupled with a nut feature of carriage (734) such that the screw gear and nut convert rotary motion from motor (736) into longitudinal motion of carriage (734) first tray drive assembly (720), and tray support platform (770). Alternatively, any other suitable components may be used.

FIGS. 10A and 10C show an example of how first tray drive assembly (730) may drive tray support platform (770) along a horizontal “y” dimension. FIG. 10A shows tray support platform (770) at a first position along the “y” dimension while FIG. 10C shows tray support platform (770) at a second position along the “y” dimension. Such selective positioning of tray support platform (770) along the “y” dimension, particularly in combination of selective positioning of tray support platform (770) along the “x” dimension as described above, may enable certain wells (782) of trays (780) to be selectively positioned in relation to sampling head assemblies (750), as will be described in greater detail below.

As noted above, and as shown in FIGS. 9-11B, fluid processing assembly (700) includes head support actuation assembly (740), which is operable to drive sampling head assemblies (750) along a vertical dimension to thereby selectively raise and lower sampling head assemblies (750) relative to trays (780). Head support actuation assembly (740) includes a head support plate (742), a pair of pneumatic cylinders (744), a pair of brackets (746), and a pair of rods (748). Sampling head assemblies (750) are secured to head support plate (742) as will be described in greater detail below. Pneumatic cylinders (744) are rigidly secured to frame (710) by brackets (746). A lower end of each rod (748) is rigidly secured to head support plate (742). An upper end of each rod (748) includes a piston (not shown) that is contained within a corresponding pneumatic cylinder (744).

Each pneumatic cylinder (744) is coupled with pneumatic assembly (714) via one or more tubes or other conduits, such that pneumatic assembly (714) is operable to communicate pressurized air to pneumatic cylinders (744) to thereby drive longitudinal translation of rods (748) relative to pneumatic cylinders (744). Pneumatic assembly (714) may be coupled with control circuit assembly (712) via one or more cables, etc., such that control circuit assembly (712) may control activation of pneumatic assembly (714). In some versions, pneumatic cylinders (744) are double-acting cylinders, such that each pneumatic cylinder (744) has a first pneumatic fitting near a lower end of pneumatic cylinder (744) and a second pneumatic fitting near an upper end of pneumatic cylinder (744). In some other versions, pneumatic cylinders (744) are single-acting cylinders.

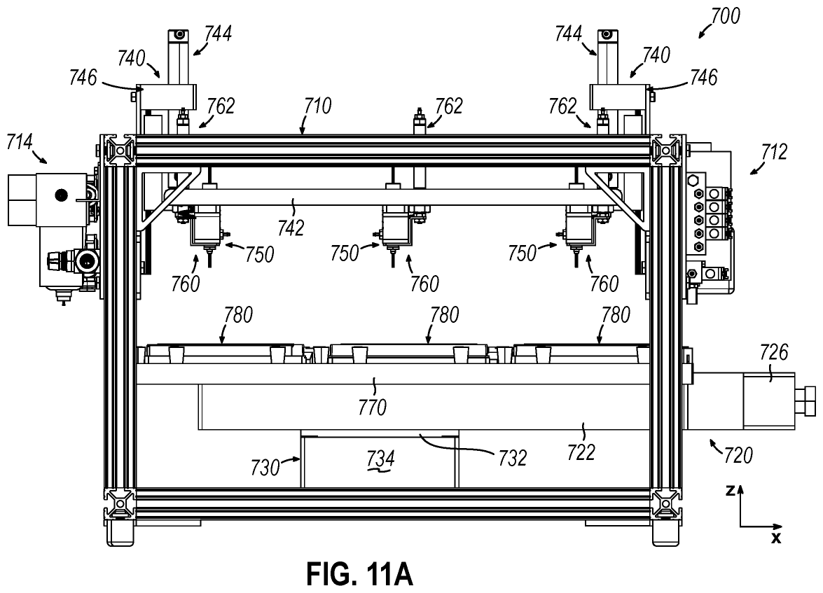

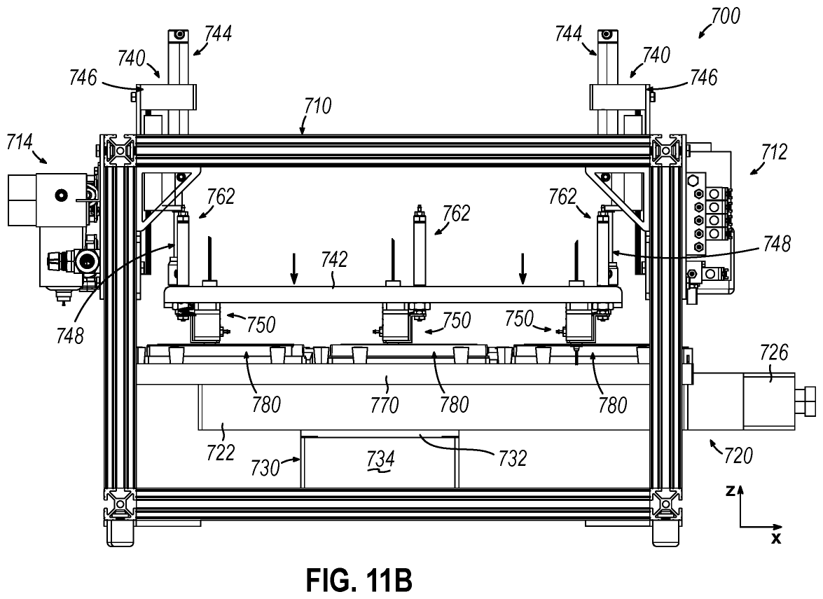

FIGS. 11A and 11B show an example of how head support actuation assembly (740) may drive sampling head assemblies (750) along a vertical “z” dimension. FIG. 11A shows head support plate (742) and sampling head assemblies (750) at a first position along the “z” dimension while FIG. 11B shows head support plate (742) and sampling head assemblies (750) at a second position along the “z” dimension. In the state shown on FIG. 11A, rods (748) are in a retracted position relative to the corresponding pneumatic cylinders (744); while in the state shown in FIG. 11B, rods (748) are in an extended position relative to the corresponding pneumatic cylinders (744). By driving sampling head assemblies (750) from the upper position shown in FIG. 11A to the lower position shown in FIG. 11B, head support actuation assembly (740) is operable to selectively place sampling head assemblies (750) in fluid communication with wells (782) of trays (780) as will be described in greater detail below.

After reaching the lower position shown in FIG. 11B and achieving the desired fluid communication between sampling head assemblies (750) and wells (782) of trays (780), head support actuation assembly (740) may return to the state shown in FIG. 11A to allow tray drive assemblies (720, 730) to drive tray support platform (770) relative to frame (710) along the horizontal plane to thereby align different wells (782) with sampling head assemblies (750). The above-described movements by tray drive assemblies (720, 730) and head support actuation assembly (740) may be carried out any suitable number of times to provide selective fluid communication between sampling head assemblies (750) and any suitable number of wells (782).

While tray drive assemblies (720, 730) are driven by motor (726, 736) in the present example, any other suitable kind of mechanisms may be used to actuate tray drive assemblies (720, 730). For instance, tray drive assemblies (720, 730) may be solenoid driven, pneumatically driven, hydraulically driven, or otherwise driven. Similarly, while head support actuation assembly (740) is pneumatically driven by pneumatic cylinders (744) and rods (748) in the present example, any other suitable kind of mechanisms may be used to actuate head support actuation assembly (740). For instance, head support actuation assembly (740) may be motor driven, solenoid driven, hydraulically driven, or otherwise driven.

### C. Examples of Features to Index Trays

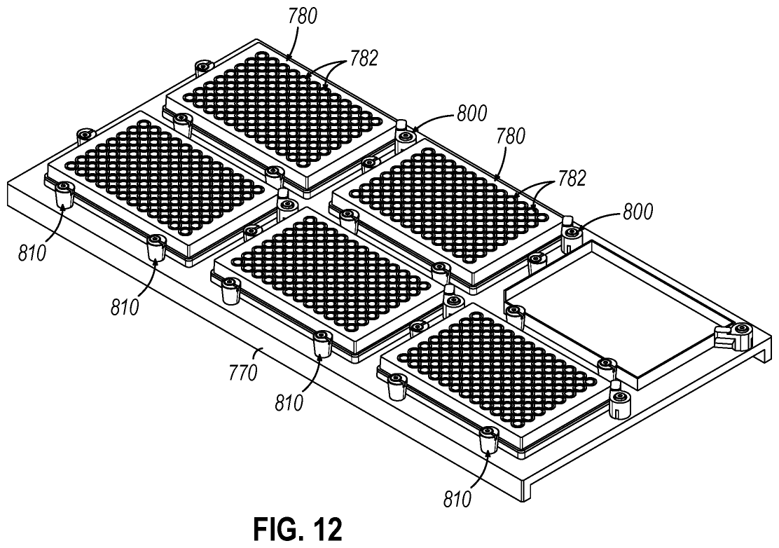

It may be desirable to provide features (e.g., poka-yoke features) that consistently provide appropriate positioning of trays (780) on tray support platform (770). This may assist in ensuring that wells (782) are appropriately positioned in relation to sampling head assemblies (750) throughout operation of fluid processing assembly (700). FIGS. 12-14 show examples of such features.

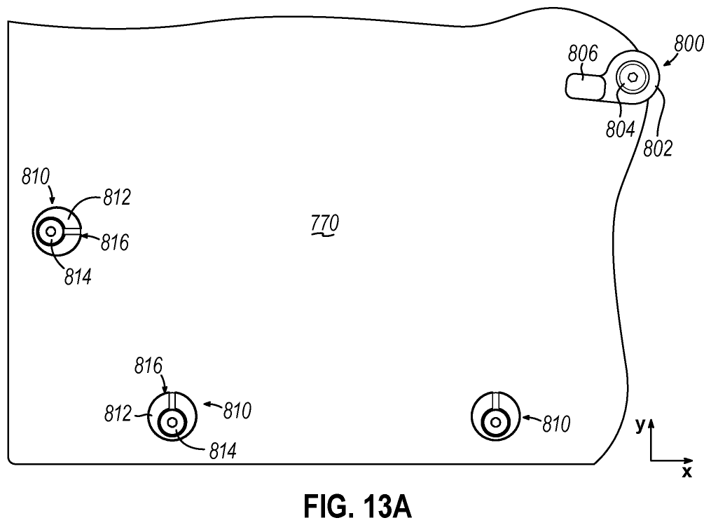

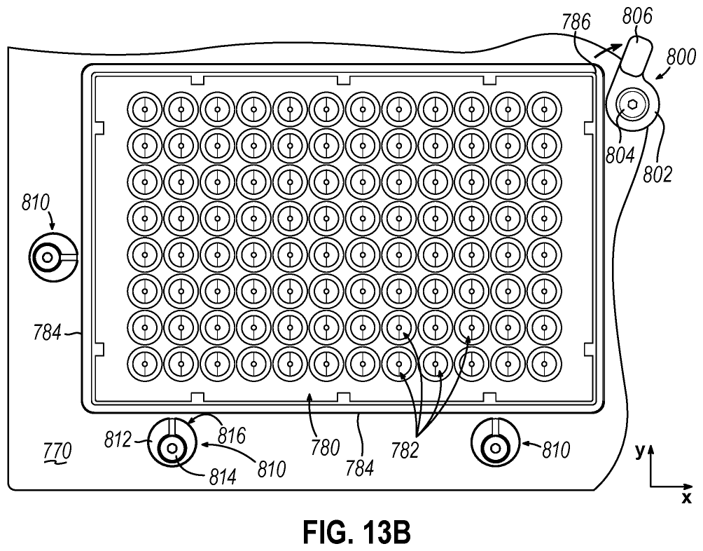

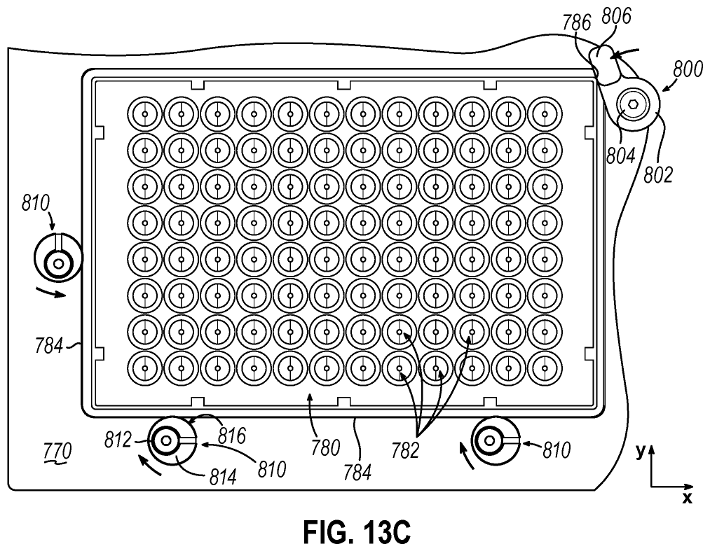

As shown in FIGS. 12-14 tray support platform (770) includes a set of first indexing features (800) and a set of second indexing features (810). Tray support platform (770) is configured such that one first indexing feature (800) and three second indexing features (810) will engage a tray (780). Each first indexing feature (800) includes a body (802) that is rotatably mounted to a post (804). Body (802) includes a tangentially extending arm (806) that is configured to engage a corner (786) of tray (780). Body (802) is configured to rotate about post (804) through an angular range of motion. A biasing member (not shown) is configured to resiliently urge body (802) toward the angular position shown in FIG. 13A. Such a biasing member of first indexing feature (800) may include a torsion spring or any other suitable component(s). The biasing member of first indexing feature (800) may have a shape memory that urges the biasing member toward a particular “neutral” shape or configuration (e.g., a straight configuration). The biasing member may be deformable away from that particular neutral shape or configuration (e.g., into a coiled shape or configuration or otherwise bent shape or configuration), though such deformation may induce stress in the material comprising the biasing member. Such stress may urge the biasing member to return to the neutral shape or configuration.

In the context of body (802), the biasing member of first indexing feature (800) may be in the neutral state in the angular position shown in FIG. 13A; and in a stressed state in angular positions other than the angular position shown in FIG. 13A. In some other variations, the biasing member of first indexing feature (800) may be prestressed when body (802) is in the angular position shown in FIG. 13A, with a mechanical stop feature resisting further rotation of body (802) in a counterclockwise direction (in the view shown in FIGS. 13A-13C) from the angular position shown in FIG. 13A. In such versions, the biasing member of first indexing feature (800) may be in a first stressed configuration when body (802) is in the angular position shown in FIG. 13A; and in a further stressed configuration when body (802) is rotated clockwise (in the view shown in FIG. 13A-13C) to other angular positions, such that the stress in the biasing member is increased when body (802) is rotated away from the angular position shown in FIG. 13A.

Regardless of whether the biasing member of first indexing feature (800) is in a neutral state in the angular position shown in FIG. 13A or a prestressed state in the angular position shown in FIG. 13A, when body (802) is rotated clockwise (in the view shown in FIG. 13A-13C) from the position shown in FIG. 13A to other angular positions, the induced or increased stress in the biasing member of first indexing feature (800) may urge body (802) to rotate counterclockwise (in the view shown in FIG. 13A-13C) toward the angular position shown in FIG. 13A. In the event that body (802) is contacting tray (780) (e.g., via arm (806)) when the biasing member of first indexing feature (800) is in a stressed state, the stress in the biasing member of first indexing feature (800) may cause body (802) to bear against tray (780). In other words, when body (802) contacts tray (780) with body (802) being in some angular position that is not the angular position shown in FIG. 13A, body (802) will resiliently bear against tray (780) as the biasing member of first indexing feature (800) resiliently urges body (802) to return to the angular position shown in FIG. 13A. Such resilient bearing of body (802) against tray (780) may in turn resiliently urge tray (780) into an appropriately indexed position along the horizontal plane (i.e., the x-y plane) as described in greater detail below.

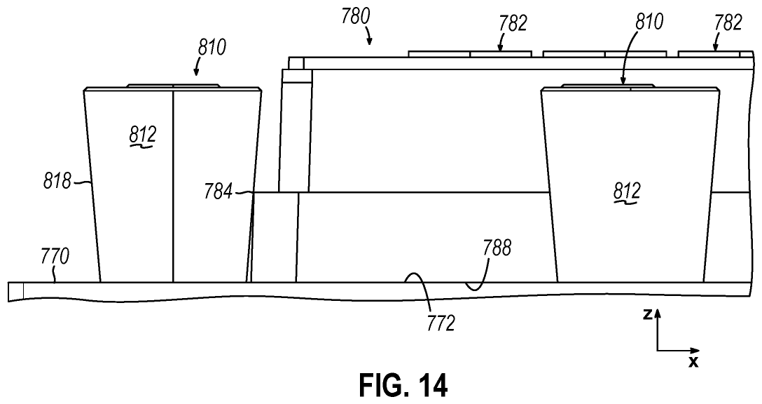

Each second indexing feature (810) includes a body (812) that is eccentrically and rotatably mounted to a post (814). Due to the eccentric mounting of body (812) to post (814), an off-axis region (816) of body (812) is laterally offset from post (814). Body (812) is configured to rotate about post (814) through an angular range of motion. A biasing member (not shown) is configured to resiliently urge body (812) toward the angular position shown in FIG. 13A. Such a biasing member may include a torsion spring or any other suitable component(s). As best seen in FIG. 14, body (812) also includes a tapered sidewall (818). Due to the taper of sidewall (818) the diameter of body (812) is smaller at upper surface (772) of tray support platform (770) than at the top of body (812).

FIGS. 13A-13C show an example of a sequence of operation of indexing features (800, 810) and a tray (780). As indicated above, FIG. 13A shows indexing features (800, 810) in a state where indexing features are not being engaged by tray (780). To load a tray (780) on tray support platform (770), the user may manipulate first indexing feature (800) to rotate first indexing feature (800) clockwise to the position shown in FIG. 13B, to thereby provide clearance for corner (786) of tray (780). The user need not necessarily position tray (780) at a precisely indexed position in the state shown in FIG. 13B. Once tray (780) has been positioned on upper surface (772) of tray support platform (770), such as in the position shown in FIG. 13B, the user may release first indexing feature (800).

When the user releases first indexing feature (800) after reaching the state shown in FIG. 13B, the resilient bias in first indexing feature (800) may urge first indexing feature (800) counterclockwise, such that arm (806) contacts corner (786) and thereby resiliently urges tray (780) into engagement with second indexing features (810) as shown in FIG. 13C. As outer edges (784) of tray (780) engage second indexing features (810), bodies (812) of second indexing features may rotate about respective posts (814). Upon reaching the state shown in FIG. 13C, the resilient bias imposed by first and second indexing features (800, 810) on tray (780) may appropriately index tray (780) along the horizontal plane (i.e., the x-y plane). In other words, in the indexed state shown in FIG. 13C, wells (782) may be appropriately positioned to receive shafts (920) of sampling heads (900) of sampling head assembly (750) as will be described in greater detail below.

In addition to assisting with indexing of tray (780) along the horizontal plane, second indexing features (810) may also assist in ensuring that tray (780) is appropriately seated against upper surface (772) of tray support platform (770). As shown in FIG. 14, the tapered configuration of sidewalls (818) may provide a camming action against outer edges (784) of tray (780) that urges the bottom edge (788) of tray (788) to fully seat on upper surface (772) of tray support platform (770). This vertical urging of tray (780) in the “z” direction may further promote appropriate indexing of wells (782) relative to shafts (920) of sampling heads (900) of sampling head assembly (750).

In some variations, second indexing features (810) do not rotate about corresponding shafts (814). In some such variations, second indexing features (810) are rigidly fixed in the angular positions shown in FIG. 13C.

In the present example, tray support platform (770) is sized and configured to receive six trays (780). Alternatively, tray support platform (770) may be sized and configured to receive any other number trays (780). In addition, tray support platform (770) and indexing features (800, 810) are sized and configured to accommodate trays (780) having 96 wells (782) per tray (780). Alternatively, tray support platform (770) and indexing features (800, 810) may be sized and configured to accommodate trays (780) having any other suitable number of wells (782) per tray (780).

### D. Example of Sampling Head Assembly

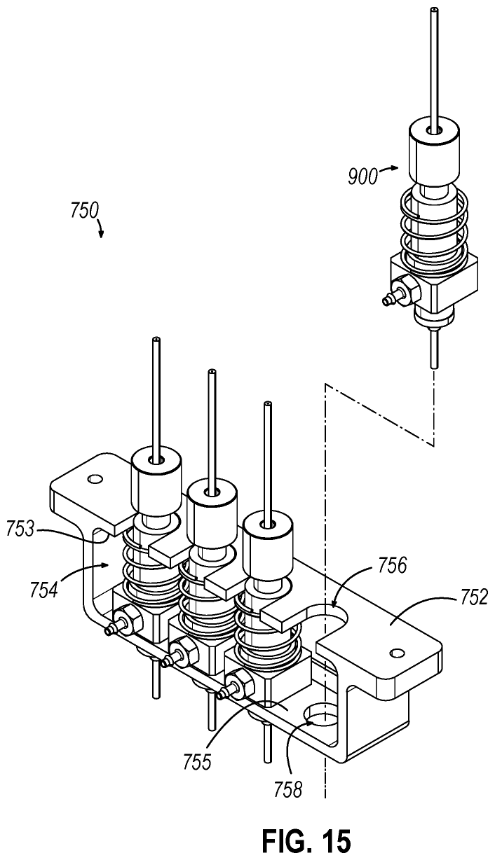

FIGS. 15-19 show components of sampling head assembly (750) in greater detail. While fluid processing assembly (700) of the present example has three sampling head assemblies (750), other variations of fluid processing assembly (700) may have more or fewer than three sampling head assemblies (750). As shown in FIG. 15, each sampling head assembly (750) of the present example includes a body (752) and four sampling heads (900). While each sampling head assembly (750) of the present example has four sampling heads (900), other variations of sampling head assembly (750) may have more or fewer than four sampling heads (900).

Body (752) is rigidly mounted to head support plate (742) of head support actuation assembly (740). Body (752) defines a cavity (754), a set of upper slots (756), and a set of lower openings (758). Cavity (754) is configured to accommodate bodies (950) of sampling heads (950), with upper portions (952) of bodies (950) passing through upper slots (756); and with lower portions (756) of bodies (950) passing through lower openings (758).

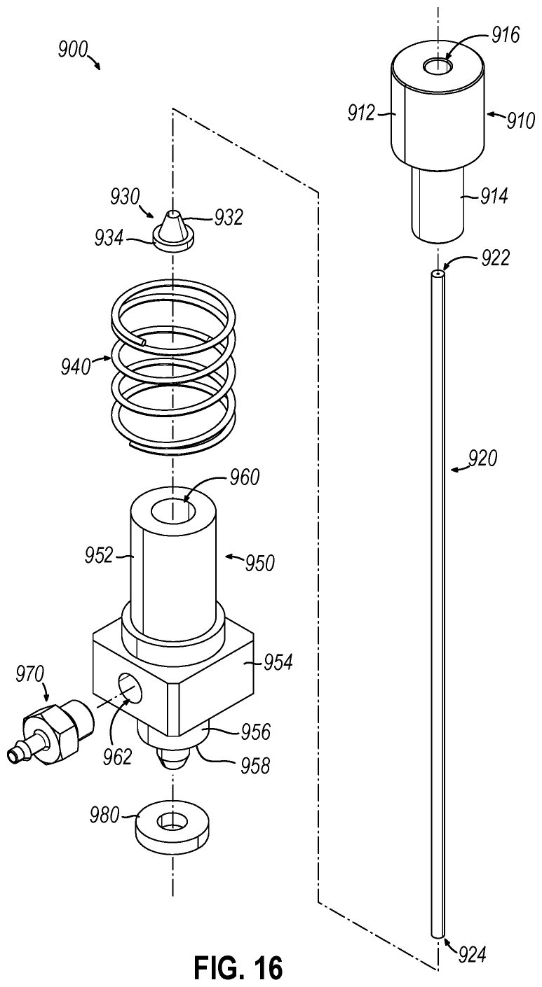

As best seen in FIG. 16, each sampling head (900) includes a compression member (910), a shaft (920), a ferrule (930), a spring (940), a body (950), a pneumatic fitting (970), and a gasket (980). Compression member (910) includes a body (912) with a threaded region (914) and a central passageway (916). Shaft (920) is hollow and includes an open upper end (922) and an open lower end (924). Ferrule (930) includes a frustoconical portion (932) and an annular base (934). Spring (940) of the present example is a coil spring, though any other suitable kind(s) of resilient member(s) may be used.

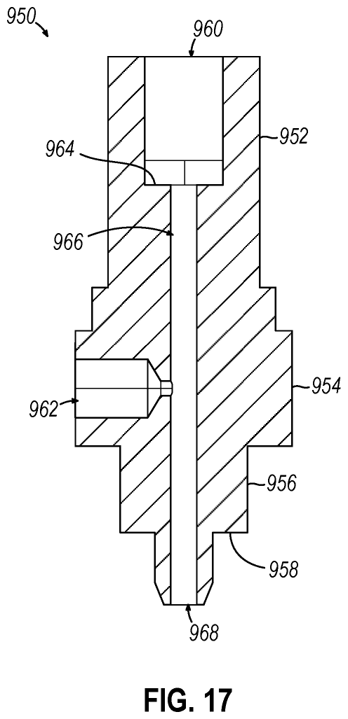

Body (950) includes a cylindraceous upper portion (952), an intermediate block portion (954), and a cylindraceous lower portion (956). As best seen in FIG. 17, upper portion (952) defines an upper passageway (960) with an internal floor (964). A main passageway (966) passes through the rest of the length of body (950), extending from internal floor (964) to a lower opening (968). A transverse passageway (962) extends through intermediate block portion (954) to main passageway (966).

Spring (940) is configured to fit about upper portion (952) of body (950). A lower end of spring (940) is positioned and configured to bear against an upper surface of intermediate block portion (954). An upper end of spring (940) is positioned and configured to bear against an upper inner surface (753) in cavity (754) of body (752). A lower surface of intermediate block portion (954) is positioned and configured to abut a lower inner surface (755) in cavity (754) of body (752). Spring (940) resiliently urges intermediate block portion (954) toward lower inner surface (755), such that the lower surface of intermediate block portion (954) may resiliently bear against lower inner surface (755). However, as will be described in greater detail below, spring (940) may also compress during operation of fluid processing assembly (700) to allow intermediate block portion (954) to travel upwardly, away from lower inner surface (755).

Pneumatic fitting (970) is configured to fit in transverse passageway (962). In some versions, pneumatic fitting (970) provides a threaded, fluid-tight fit in transverse passageway (962). Pneumatic fitting (970) is further configured to couple with a flexible tube or other conduit, such that pneumatic fitting (970) may be placed in fluid communication with pneumatic assembly (714) via pneumatic fitting (970). This may enable pneumatic assembly (714) to communicate pressurized air to main passageway (966) via pneumatic fitting (970) and transverse passageway (962), as will be described in greater detail below.

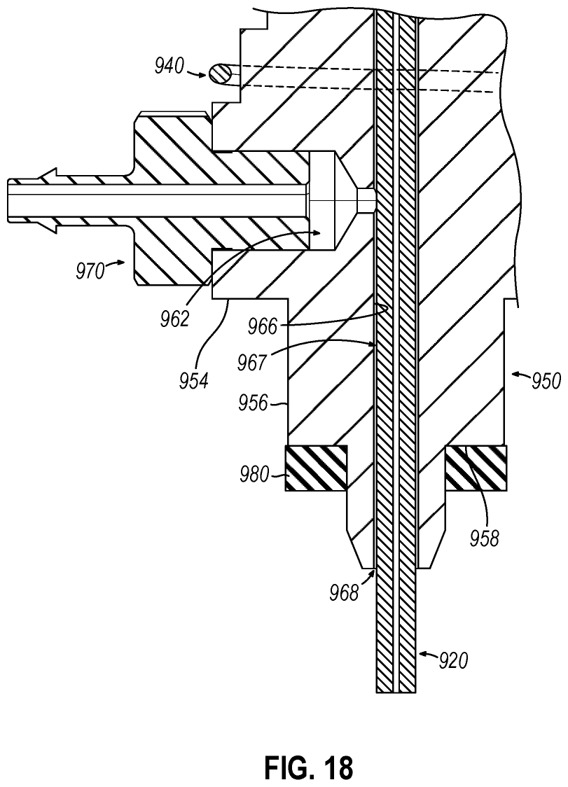

Gasket (980) is an elastomeric annular member in the present example. Gasket (980) may comprise rubber and/or any other suitable material(s). Gasket (980) is configured to seat against a lower surface (958) of lower portion (956) of body (950), as best seen in FIG. 18. Gasket (980) is also configured to provide a fluid tight seal against the top region of a well (782) of tray (780) during operation of fluid processing assembly (700), as will be described in greater detail below.

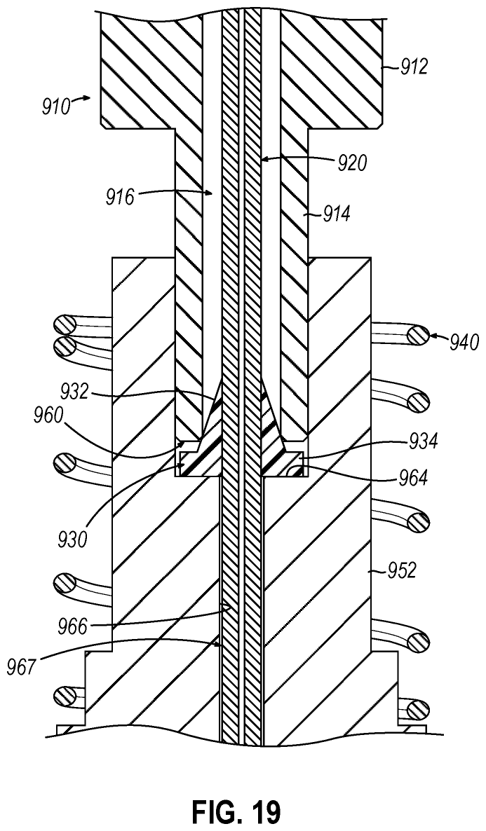

Shaft (920) is coaxially disposed within central passageway (916), ferrule (930), and main passageway (966). As shown in FIGS. 18-19, main passageway (966) has a diameter that is larger than the outer diameter of shaft (920), such that a gap (967) is defined between the inner surface of main passageway (966) and the outer surface of shaft (920). This gap (967) extends distally to lower opening (968). Transverse passageway (962) is in fluid communication with gap (967), such that transverse passageway (962) and gap (967) together define a passageway for pressurized air to flow from pneumatic fitting (970) to lower opening (968). As shown in FIG. 19, ferrule (930) is positioned and configured to effectively seal the upper end of gap (967). Annular base (934) of ferrule (930) is sized and configured to seat against a floor (964) of upper passageway (966). With shaft (920) disposed in ferrule (930), and with annular base (934) seated against floor (964), threaded region (914) of compression member (910) may be inserted into upper passageway (966) and rotated such that threading of threaded region (914) engages complementary threading formed in upper passageway (966). Compression member (910) may be rotated relative to body (950) until compression member (910) reaches the position shown in FIG. 19, at which point the distal end of compression member (910) may bear against frustoconical portion (932) of ferrule (930) and thereby deform frustoconical portion (932) against shaft (920). The compressed and deformed ferrule (930) may thus form a seal against shaft (920), thereby preventing pressurized air from escaping upwardly from gap (967). Alternatively, any other suitable components may be used to seal the upper end of gap (967).

Upper end (922) of each shaft (920) may be coupled with a corresponding flexible tube (not shown) or other fluid conduit. These flexible tubes or other conduits that are coupled with upper ends (922) of shafts (920) may form fluid communication pathways from fluid processing assembly (570) to reagent storage frame (107) of system (100). In other words, the flexible tubes or other conduits that are coupled with upper ends (922) of shafts (920) may collectively serve as fluid communication pathway (532) in arrangements such as those shown in FIG. 6; or as fluid communication pathway (573) in arrangements such as those shown in FIG. 7. Lower end (924) of each shaft (920) is configured to fit in a well (782) of tray (780). Lower ends (924) may thus be used to communicate fluid from or to wells (782). With ends (922, 924) being open and shaft (920) being hollow (920), shafts (920) and the flexible tubes or other conduits that are coupled with upper ends (922) of shafts (920) may together form fluid communication pathways like fluid communication pathways (634, 644, 654) between trays (780) and another fluid processing assembly like fluid processing assembly (610).

### E. Example of Tray Engagement Assembly

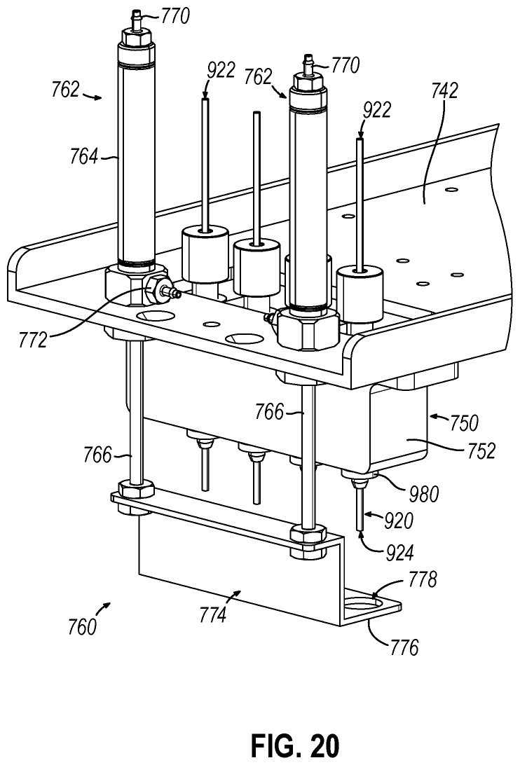

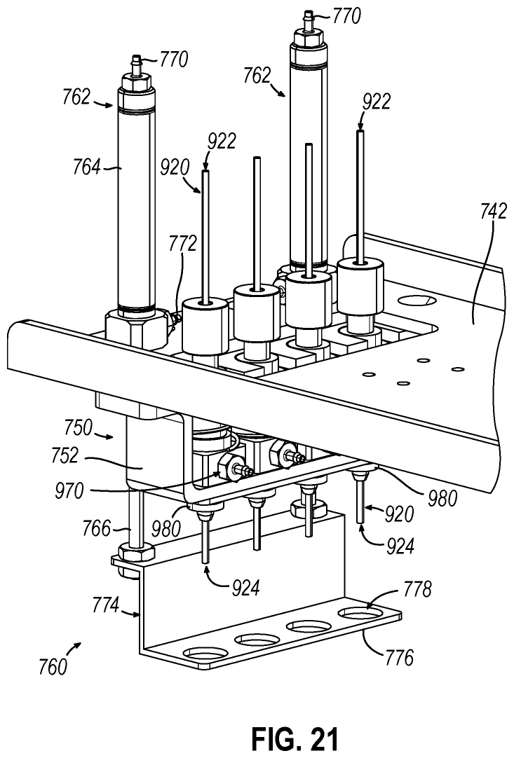

As noted above, tray engagement assemblies (760) are operable to selectively engage trays (780) during at least part of the vertical range of travel of sampling head assemblies (750) relative to trays (780). As shown in FIGS. 20-21, each tray engagement assembly (760) includes a pair of pneumatic actuators (762) and a tray engagement plate (774). Each pneumatic actuator (762) includes a pneumatic cylinder (764) and a rod (766). Pneumatic cylinders (764) are rigidly secured to head support plate (742). A lower end of each rod (766) is rigidly secured to tray engagement plate (774). An upper end of each rod (766) includes a piston (not shown) that is contained within a corresponding pneumatic cylinder (764). Each pneumatic cylinder (764) has an upper pneumatic fitting (770) and a lower pneumatic fitting (772). Pneumatic fittings (770, 772) are coupled with pneumatic assembly (714) via one or more tubes or other conduits, such that pneumatic assembly (714) is operable to communicate pressurized air to pneumatic cylinders (764) to thereby drive longitudinal translation of rods (766) relative to pneumatic cylinders (764). In the present example, pneumatic cylinders (764) are double-acting cylinders, such that rods (766) may be actively pneumatically driven upwardly relative to pneumatic cylinders (764) and actively pneumatically driven downwardly relative to pneumatic cylinders (764). In some other versions, pneumatic cylinders (764) are single-acting cylinders.

Tray engagement plate (774) includes a horizontally extending foot (776), which is configured to engage tray (780) as described in greater detail below. Foot (776) defines a plurality of openings (778) that correspond with sampling heads (900). Openings (778) are sized and positioned to accommodate shafts (920), gaskets (980), and lower portions (956) of bodies (950).

While tray engagement assemblies (760) are pneumatically driven by pneumatic cylinders (764) and rods (766) in the present example, any other suitable kind of mechanisms may be used to actuate tray engagement assemblies (760). For instance, tray engagement assemblies (760) may be motor driven, solenoid driven, hydraulically driven, or otherwise driven. In some versions, tray engagement assemblies (760) are omitted.

### F. Example of Sequence of Engaging Tray

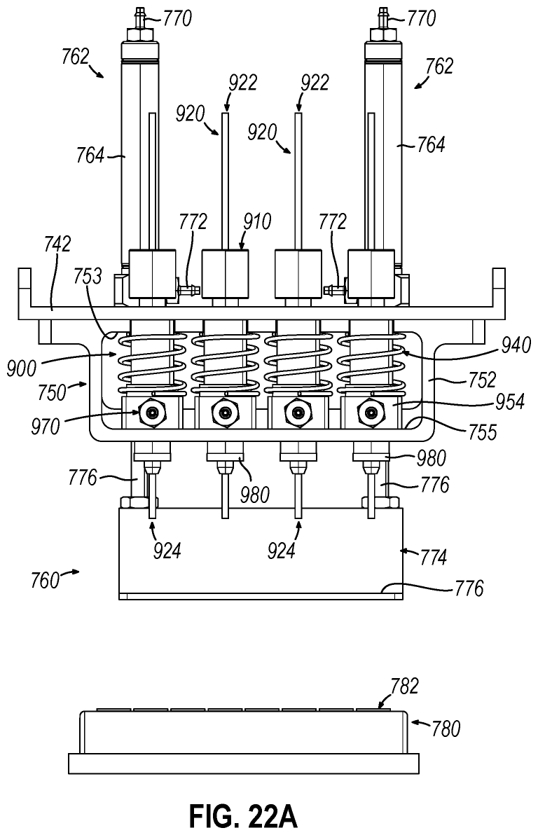

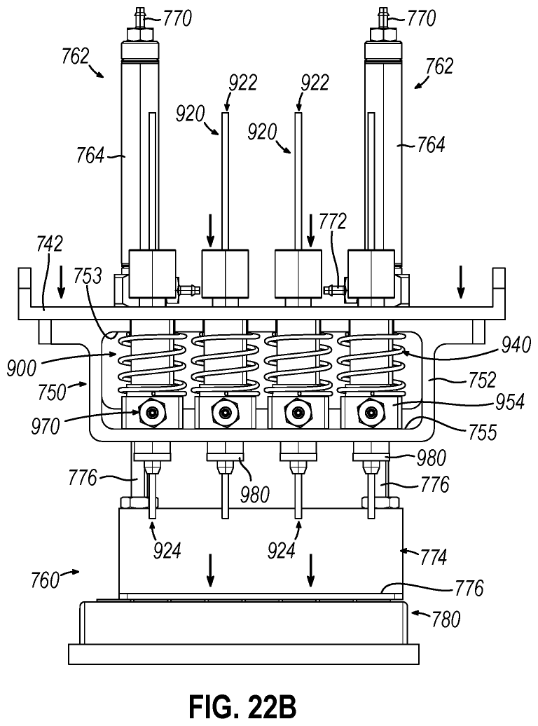

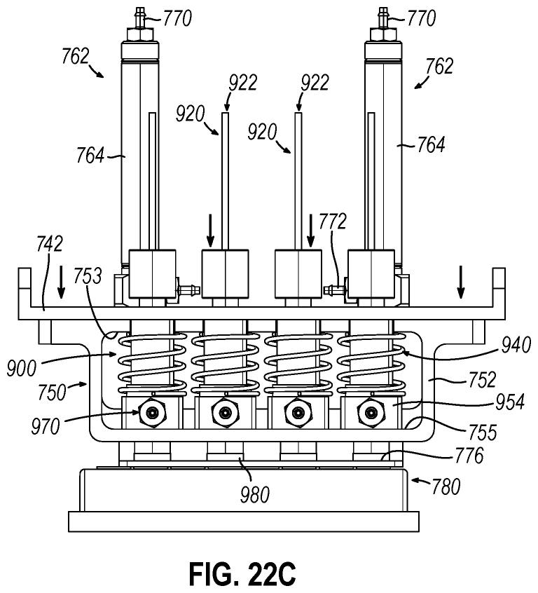

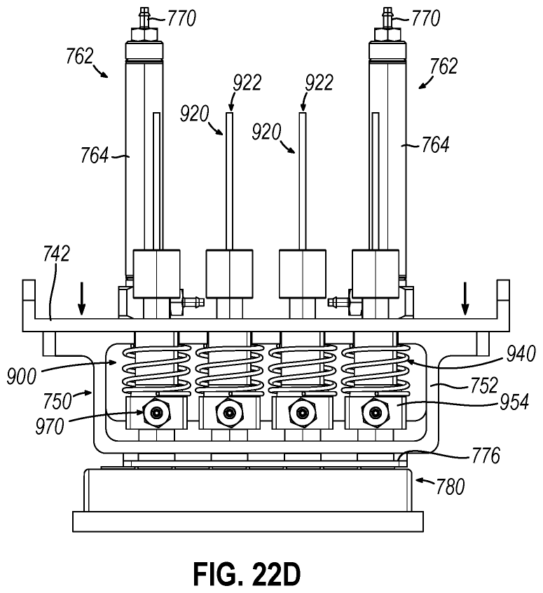

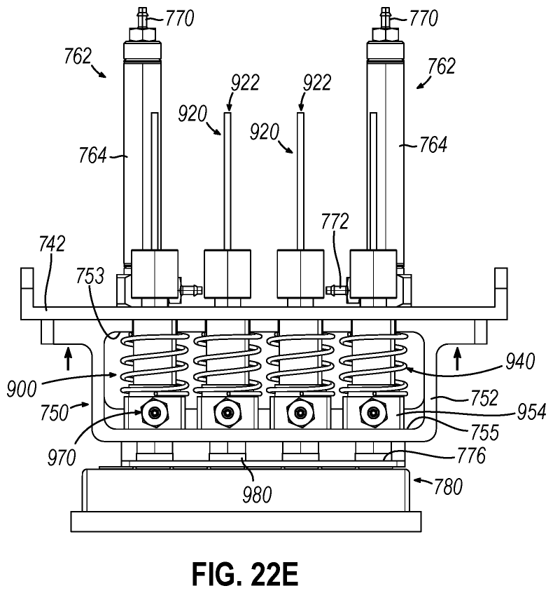

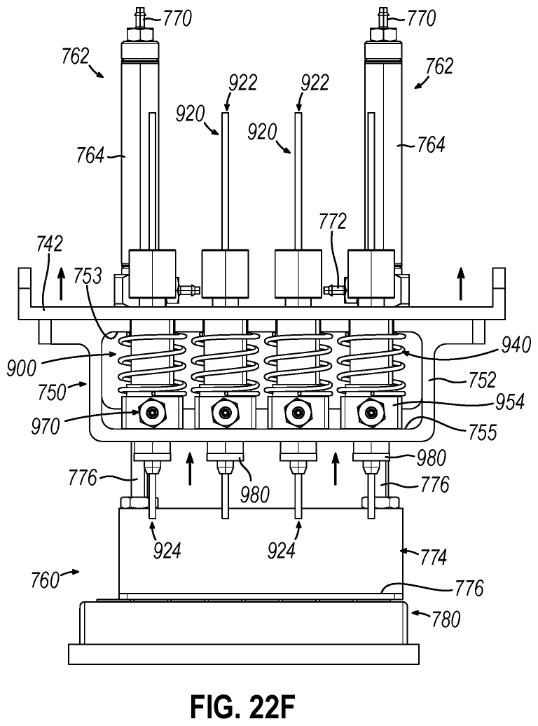

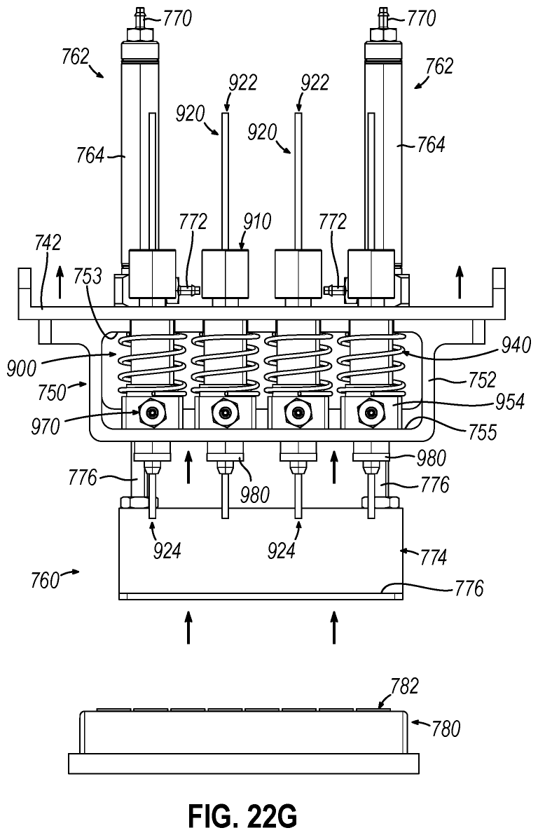

FIGS. 22A-22G show an example of a sequence of operational states of head support actuation assembly (740), sampling head assemblies (750), and tray engagement assemblies (760). While only one sampling head assembly (750) and tray engagement assembly (760) is depicted in FIGS. 22A-22G, the sampling head assemblies (750) and tray engagement assemblies (760) of fluid processing assembly (700) may all synchronously operate through the stages shown in FIGS. 22A-22G.

In the stage shown in FIG. 22A, of head support actuation assembly (740) is in an upper position, such that sampling head assembly (750) is spaced vertically over tray (780). Tray engagement assembly (760) is on a configuration where tray engagement plate (774) is in a lowered position relative to sampling head assembly (750), yet tray engagement plate (774) is spaced vertically over tray (780). Thus, no portions of sampling head assembly (750) or tray engagement assembly (760) are engaged with tray (780) at this stage. While head support actuation assembly (740), sampling head assembly (750), and tray engagement assembly (760) are in the state shown in FIG. 22A, tray drive assemblies (720, 730) may be actuated as described above with reference to FIGS. 10A-10C to appropriately position certain wells (782) directly under corresponding sampling heads (900).

Once the target wells (782) are appropriately positioned under corresponding sampling heads (900), head support actuation assembly (740) may be actuated to drive sampling head assembly (750) and tray engagement assembly (760) downwardly toward tray (780) to reach the state shown in FIG. 22B. During the transition from the state shown in FIG. 22A to the state shown in FIG. 22B, sampling head assembly (750) and tray engagement assembly (760) may travel downwardly together in unison, such that tray engagement assembly (760) does not move relative to sampling head assembly (750). The actuation of head support actuation assembly (740), to reach the state shown in FIG. 22B, may include activating pneumatic assembly (714) to drive pressurized air to pneumatic cylinders (744) (which are not shown in FIGS. 22A-22G). Upon reaching the state shown in FIG. 22B, foot (776) of tray engagement plate (774) engages the top of tray (780), thereby assisting indexing features (800, 810) in maintaining the position of tray (780) on tray support platform (770) during some subsequent stages of operation.

After reaching the state shown in FIG. 22B, head support actuation assembly (740) may be further actuated to drive sampling head assembly (750) downwardly toward tray (780) to reach the state shown in FIG. 22C. As head support actuation assembly (740) drives sampling head assembly (750) downwardly, tray engagement assembly (760) may be actuated to maintain the vertical position of tray engagement plate (774) relative to tray (780). Tray engagement plate (774) may thus move upwardly relative to sampling head assembly (750) as sampling head assembly (750) moves downwardly relative to tray (780), resulting in no vertical movement of tray engagement plate (774) relative to tray (780) during the transition from the state shown in FIG. 22B to the state shown in FIG. 22C. This actuation of tray engagement assembly (760) may include activating pneumatic assembly (714) to drive pressurized air to lower pneumatic fittings (772) of pneumatic cylinders (764).

As sampling head assembly (750) transitions from the state shown in FIG. 22B to the state shown in FIG. 22C, lower ends (924) of shafts (920) enter the targeted wells (782) of trays (780). In scenarios where wells (782) contain fluid, lower ends (924) may be disposed in the fluid at the state shown in FIG. 22B. In some versions, tray (780) includes a cover member (e.g., film, plastic sheet, foil sheet, etc.) that is used to prevent evaporation and/or contamination of the contents of wells (782). In such versions, lower ends (924) of shafts (920) may penetrate such a cover member to enter wells (782) during the transition from the state shown in FIG. 22B to the state shown in FIG. 22C. In some such versions, lower ends (924) include sharp tips, cutting edges, or other features that assist in penetration of the cover member over wells (782) during the transition from the state shown in FIG. 22B to the state shown in FIG. 22C. Such penetration assistance features are optional.

Upon reaching the state shown in FIG. 22C, gaskets (980) of sampling heads (900) may contact upper surfaces surrounding corresponding wells (782). To promote a fluid-tight seal at such an interface between gaskets (980) and the upper surfaces surrounding corresponding wells (782), head support actuation assembly (740) may be further actuated to drive sampling head assembly (750) downwardly toward tray (780) to reach the state shown in FIG. 22D. As head support actuation assembly (740) drives sampling head assembly (750) downwardly, tray engagement assembly (760) may be actuated to maintain the vertical position of tray engagement plate (774) relative to tray (780). Tray engagement plate (774) may thus move upwardly relative to sampling head assembly (750) as sampling head assembly (750) moves downwardly relative to tray (780), resulting in no vertical movement of tray engagement plate (774) relative to tray (780) during the transition from the state shown in FIG. 22C to the state shown in FIG. 22D.

During the transition from the state shown in FIG. 22C to the state shown in FIG. 22D, gaskets (980) may bear against the upper surfaces around wells (782), such that the upper surfaces around wells (782) provide an upward opposing force against sampling head (900). This opposing force may cause springs (940) to compress, such that the lower surfaces of intermediate block portions (954) disengage lower inner surface (755) of body (752). In other words, body (752) continues to travel downwardly with head support plate (742) during the transition from the state shown in FIG. 22C to the state shown in FIG. 22D; while sampling heads (900) remain vertically stationary during this transition. In the state shown in FIG. 22D, compressed springs (940) resiliently urge gaskets (980) against the upper surfaces around wells (782). This resilient urging of gaskets (980) against the upper surfaces around wells (782) may promote a fluid-tight seal at the interface between gaskets (980) and the upper surfaces around wells (782).

With gaskets (980) being firmly sealed against the upper surfaces around wells (782) during the state shown in FIG. 22D, fluid may be communicated from or to the wells (782) via shafts (920). In scenarios where fluid is communicated from wells (782) to shafts (920), such fluid communication may be achieved by activating pneumatic assembly (914) to communicate pressurized air to gap (967) via pneumatic fitting (970) and transverse passageway (962). This pressurized air may exit sampling head (900) via lower opening (968) and thereby pneumatically bear against the surface of fluid contained in well (782). This pressurization in the air over the fluid may drive the fluid upwardly through the lumen defined by shaft (920). The fluid from well (782) may exit upper end (922) of shaft (920) and then be communicated along the flexible tube or other conduit that is coupled with upper end (922) of shaft (920), ultimately reaching a destination such as reagent storage frame (107), fluid processing assembly (514), fluid processing assembly (564), fluid processing assembly (610), etc.

In scenarios where fluid is communicated to wells (782) from shafts (920), such fluid communication may be achieved by activating a source such as reagent storage frame (107), fluid processing assembly (514), fluid processing assembly (564), fluid processing assembly (610), etc. to drive fluid along the flexible tube or other conduit that is coupled with upper end (922) of shaft (920). The fluid may enter the lumen of shaft (920) via upper end (922) and exit shaft (920) via lower end (924), thereby entering well (782). During this process, gap (967), transverse passageway (962), and pneumatic fitting (970) may be vented to atmosphere, thereby allowing air to escape from well (782) as fluid is deposited in well (782).

After the desired fluid has been communicated from or to wells (782), the above-described process may be reversed. As part of this reversal, head support actuation assembly (740) may be actuated to drive sampling head assembly (750) upwardly toward tray (780) to reach the state shown in FIG. 22E. Simultaneously, tray engagement assembly (760) may be actuated to maintain the vertical position of tray engagement plate (774) relative to tray (780). Tray engagement plate (774) may thus move downwardly relative to sampling head assembly (750) as sampling head assembly (750) moves upwardly relative to tray (780), resulting in no vertical movement of tray engagement plate (774) relative to tray (780) during the transition from the state shown in FIG. 22D to the state shown in FIG. 22E. Springs (940) may also drive intermediate block portions (954) back into engagement with lower inner surface (755) of body (752) during the transition from the state shown in FIG. 22D to the state shown in FIG. 22E.

Head support actuation assembly (740) may continue to be actuated to drive sampling head assembly (750) upwardly toward tray (780) to reach the state shown in FIG. 22F. Simultaneously, tray engagement assembly (760) may continue to be actuated to maintain the vertical position of tray engagement plate (774) relative to tray (780). Tray engagement plate (774) may thus continue to move downwardly relative to sampling head assembly (750) as sampling head assembly (750) moves upwardly relative to tray (780), resulting in no vertical movement of tray engagement plate (774) relative to tray (780) during the transition from the state shown in FIG. 22E to the state shown in FIG. 22F. Shafts (920) may exit wells (782) during the transition from the state shown in FIG. 22E to the state shown in FIG. 22F, such that lower ends (924) of shafts (920) are spaced above tray (780) in the state shown in FIG. 22F. In versions where a cover member is disposed over tray (780), and lower ends (924) had penetrated the cover member during the transition from the state shown in FIG. 22B to the state shown in FIG. 22C, the continued engagement between foot (776) and tray (780) may assist in preventing friction between shafts (920) and the cover member from causing tray (780) to lift upwardly as shafts (920) transition from the position shown in FIG. 22E to the position shown in FIG. 22F. While FIG. 22F shows foot (776) remaining engaged with tray (780) after lower ends (924) have substantially cleared the top of tray (780), foot (776) may disengage tray (780) sooner in the process (e.g., immediately after lower ends (924) have cleared the top of tray (780).

After lower ends (924) have sufficiently cleared the top of tray (780), head support actuation assembly (740) may be actuated to drive sampling head assembly (750) and tray engagement assembly (760) upwardly away from tray (780) to reach the state shown in FIG. 22G. During the transition from the state shown in FIG. 22F to the state shown in FIG. 22G, sampling head assembly (750) and tray engagement assembly (760) may travel upwardly together in unison, such that tray engagement assembly (760) does not move relative to sampling head assembly (750). After reaching the state shown in FIG. 22G, tray drive assemblies (720, 730) may be actuated as described above with reference to FIGS. 10A-10C to appropriately position other wells (782) directly under corresponding sampling heads (900). The process depicted in FIGS. 22A-22G may again be repeated until the desired number of wells (782) have been addressed.

### G. Example of Method of Utilizing Automated Fluid Delivery System

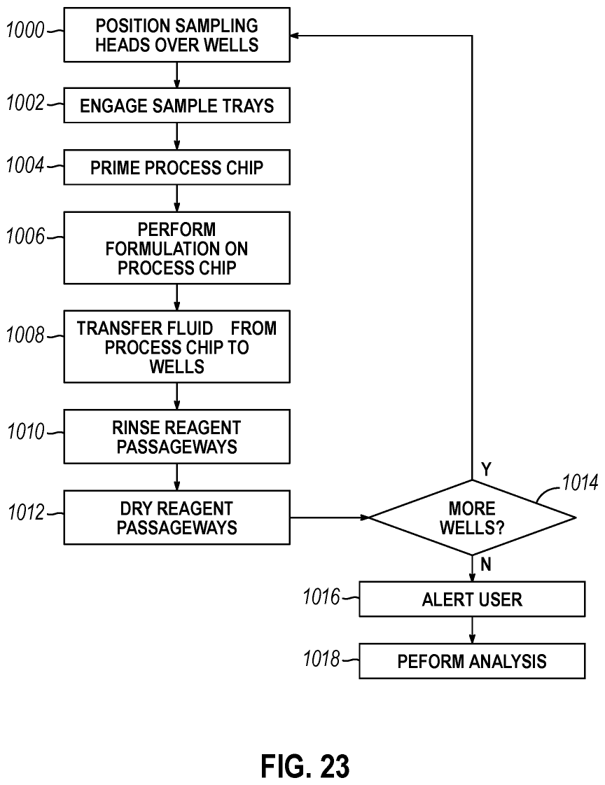

FIG. 23 depicts an example of a method of operation that may be carried out using fluid processing assembly (700) in any of the various systems (500, 550, 600) described above. In some versions, this method is carried out for screening purposes, to determine which combination of variables yield the most suitable encapsulated mRNA through a formulation process on a process chip (516, 566, 612) like process chip (400). Such variables may include, but are not necessarily limited to, reagent types, buffer compositions, DV formulations, reagent concentrations, reagent mass ratios, processing temperatures, fluid flow rates, fluid flow rate ratios, etc. Alternatively, this method may be used for any other suitable purpose(s).

In the context of a screening use, the process may begin with fluid processing assembly (700) already having one, two, or more trays (780) that are loaded with reagents securely positioned on tray support platform (770) as described above with reference to FIGS. 13A-13C. Sampling head assemblies (750) may be positioned over targeted wells (782), as shown in block (1000) of FIG. 23. This positioning may include actuating tray drive assemblies (720, 730) as described above with reference to FIGS. 10A-10C to appropriately position targeted wells (782) directly under corresponding sampling heads (900).

With wells (782) appropriately positioned in relation to sampling head assemblies (750), head support actuation assembly (740) and tray engagement assembly (760) may be actuated to engage trays, as shown in block (1002) of FIG. 23. This engagement may include the various states of operation shown in FIGS. 22A-22D and described above, with shafts (920) being suitably positioned in the targeted wells (782), and with gaskets (980) providing fluid-tight seals with the upper surfaces surrounding wells (782).

Next, the process may include priming fluid passageways on the process chip (516, 566, 612), as shown in block (1004) of FIG. 23. In arrangements such as those shown in FIG. 6, this priming may include activating sampling heads (900) to drive reagent fluids from wells (782) toward process chip (516) via fluid communication pathway (532), fluid processing assembly (514), and fluid communication pathway (515). In arrangements such as those shown in FIG. 7, this priming may include activating sampling heads (900) to drive reagent fluids from wells (782) toward process chip (566) via fluid communication pathway (573), fluid processing assembly (564), and fluid communication pathway (565). In arrangements such as those shown in FIG. 8, this priming may include activating sampling heads (900) to drive reagent fluids from wells (642, 652, 782) toward process chip (612) via fluid communication pathways (644, 654) and fluid processing assemblies (610, 614). The priming may also include activating one or both of fluid processing assemblies (610, 614) to drive reagent fluid (e.g., buffer) from vial (602) toward process chip (612) via fluid communication pathway (604) and fluid processing assemblies (610, 614). In some versions, the priming process includes driving the fluid at a pressure of approximately 0.3 psi. In addition, the priming process may include driving the fluid at a flow rate ranging from approximately 100 microliters per minute to approximately 400 microliters per minute.

In some versions, the priming process represented by block (1004) in FIG. 23 is automated. In some such versions, sampling heads (900) are automatically activated to drive reagent fluids from wells (782) as described above after trays (780) have been suitably engaged (as represented by block (1002) in FIG. 23), until such reagent fluids reach a predetermined location on process chip (516, 566, 612). Simultaneously, one or both of fluid processing assemblies (610, 614) may be automatically activated to drive reagent fluid from vial (602). Once the reagent fluids reach the predetermined location on process chip (516, 566, 612), the fluid communication may cease until further input is provided. In some such versions, one or more sensors (e.g., sensors (105)) are used to track fluid movement on process chip (516, 566, 612), such that the one or more sensors may communicate the presence of the reagent fluids in the predetermined location to a controller (e.g., controller (121, 512, 522, 562)); and such that the controller may then automatically stop further communication of reagent fluid until further input is provided.

In some versions where process chip (516, 566, 612) is configured like process chip (400), the predetermined locations to monitor for auto-priming from wells (782) may be located along fluid channels (402a, 402b), such that the reagent fluid flow may be at least temporarily ceased before the reagent fluid flows through first mixing chamber (430). In versions where process chip (516, 566, 612) is configured like process chip (400) and a separate vial (602) is used to provide a buffer fluid, the predetermined location to monitor for auto-priming from vial (602) may be located along fluid channel (402c), such that the buffer fluid flow may be at least temporarily ceased before the buffer fluid flows through second mixing chamber (440). In versions where a controller (e.g., controller (121, 512, 522, 562)) automatically stops further communication of reagent fluid in response to the fluid reaching the predetermined location, such automatic stoppage may include automatically transitioning valves (424a, 424b, 424c) to a closed state.

In versions providing auto-priming where the controller automatically stops further communication of reagent fluid in a primed fluid channel (402a, 402b, 402c) until further input is provided, such further input may include a user input. For instance, the controller may notify the user (e.g., via user interface (123)) that all the appropriate fluid channels (402a, 402b, 402c) within process chip (400, 516, 566, 612) have been suitably primed, then await user input (e.g., approval) before moving forward with subsequent stages of the process. As another variation, the controller may track priming of all fluid channels (402a, 402b, 402c) within process chip (516, 566, 612), and then automatically proceed with subsequent stages in the process after controller has determined that all the appropriate fluid channels (402a, 402b, 402c) within process chip (400, 516, 566, 612) have been suitably primed.

Once process chip (516, 566, 612) has been suitably primed, the process may continue with formulation being performed on process chip (516, 566, 612), as represented by block (1006) in FIG. 23. This formulation process may be carried out in accordance with the above description referencing blocks (340, 350) of FIG. 4 to yield encapsulated mRNA (e.g., in the form of ANPs). In some versions, the formulation process may be completed in less than 10 milliseconds. The fluid containing the encapsulated mRNA created through the formulation process may be communicated to appropriate wells (782) in tray (780), as represented by block (1008) in FIG. 23.

In arrangements such as those shown in FIG. 6, the communication of fluid containing the encapsulated mRNA to wells (782) may include activating process chip (516), fluid processing assembly (514), and/or fluid processing assembly (524) to drive the fluid containing the encapsulated mRNA to appropriate wells (782) in tray (780) via fluid communication pathways (515, 532) and shafts (920). In arrangements such as those shown in FIG. 7, this communication of fluid containing the encapsulated mRNA to wells (782) may include activating process chip (566), fluid processing assembly (564), and/or fluid processing assembly (570) to drive the fluid containing the encapsulated mRNA to appropriate wells (782) in tray (780) via fluid communication pathways (565, 573) and shafts (920). In arrangements such as those shown in FIG. 8, this communication of fluid containing the encapsulated mRNA to wells (632, 782) may include activating process chip (612), fluid processing assembly (610), and/or fluid processing assembly (614) to drive the fluid containing the encapsulated mRNA to appropriate wells (632, 782) in tray (630, 780) via fluid communication pathway (634), which may include shafts (920).

While the formulation and collection stages are shown in separate blocks (1006, 1008) in FIG. 23, these stages may in fact overlap in time. For instance, reagent fluids may be communicated from corresponding wells (782) in tray (780) while fluid containing encapsulated mRNA is being communicated to other wells (782) in tray. In the context of an arrangement such as that shown in FIG. 8, shafts (920) of sampling heads (900) forming part of fluid communication pathway (634) may be disposed in wells (632) while sampling heads (900) forming part of fluid communication pathway (644) are disposed in wells (642); and while sampling heads (900) forming part of fluid communication pathway (654) are disposed in wells (652).

To conclude the communication of encapsulated mRNA to appropriate wells (782) in tray (780), as represented by block (1008) of FIG. 23, a purging volume of air may be communicated through shafts (920) that had been used to collect reagent fluids; and the other fluid communication components that are downstream of these reagent collection shafts (920), including corresponding passageways in process chip (400, 516, 566, 612). In some versions of arrangements such as those shown in FIG. 8, this purge may be accomplished after reagent fluid has been evacuated from wells (662, 672), such that further communication of pressurized air via pneumatic fittings (970) will eventually reach shafts (920) of the sampling heads (900) forming part of fluid communication pathways (644, 654). The pressurized air may flow through these reagent collection shafts (920) and the other fluid communication components that are downstream of these reagent collection shafts (920), eventually exiting shafts (920) of sampling heads (900) forming part of fluid communication pathway (634).

After the fluid containing the encapsulated mRNA has been communicated to appropriate wells (782) in tray (780), including the air purge described above, the process may then include rinsing of reagent passageways within fluid processing assembly (700), as represented by block (1010) of FIG. 23. As part of the rinsing procedure, fluid processing assembly (700) may be actuated to proceed through the operational states shown in FIGS. 22E-22G as described above, then actuate tray drive assemblies (720, 730) to appropriately position wells (782) containing rinse fluid under corresponding sampling heads (900). In the context of the arrangement shown in FIG. 8, this may include positioning wells (662) of tray (660) under corresponding sampling heads (900) of fluid communication pathway (644); and positioning wells (672) of tray (670) under corresponding sampling heads (900) of fluid communication pathway (654).

Once wells (782) containing rinse fluid are appropriately positioned in relation to sampling heads (900), fluid processing assembly (700) may be actuated to proceed through the operational states shown in FIGS. 22A-22D as described above to place shafts (920) in sealed fluid communication with the rinse fluid. Pneumatic fittings (970) may be pressurized as described above to drive the rinse fluid through shafts (920) that had been used to collect reagent fluids; and the other fluid communication components that are downstream of these reagent collection shafts (920), including corresponding passageways in process chip (400, 516, 566, 612). The rinse fluid may thus rinse these reagent collection shafts (920) and the other fluid communication components that are downstream of these reagent collection shafts (920). In the context of an arrangement such as that shown in FIG. 8, the rinse fluid may be collected via shafts (920) of fluid communication pathways (634, 644) that had been used to collect reagents during the priming and formulation stages represented by blocks (1004, 1006) of FIG. 23.

During the rinsing process represented by block (1010) of FIG. 23, the waste fluid generated through rinsing may be communicated to dedicated wells (782) in a tray (780). For instance, in system (600) described above, a tray (780) may be designated as a waste tray (680), such that wells (682) are dedicated to receiving waste. In the arrangement of system (600) shown in FIG. 8, shafts (920) of sampling heads (900) forming part of fluid communication pathway (634) may be disposed in wells (682) while sampling heads (900) forming part of fluid communication pathway (644) are disposed in wells (672); and while sampling heads (900) forming part of fluid communication pathway (654) are disposed in wells (662). Thus, wells (682) may readily receive waste fluid via fluid communication pathway (634) while rinse fluid is communicated from wells (662, 672) via fluid communication pathways (644, 654).

After a suitable volume of rinse fluid has been communicated through the reagent collection shafts (920) and the other fluid communication components that are downstream of the reagent collection shafts (920), these fluid passageways may be dried, as represented by block (1012) of FIG. 23. This drying process may include communicating pressurized air through the reagent collection shafts (920) and the other fluid communication components that are downstream of the reagent collection shafts (920), including corresponding passageways in process chip (400, 516, 566, 612). In some versions of arrangements such as those shown in FIG. 8, this drying may be accomplished after rinse fluid has been evacuated from wells (662, 672), such that further communication of pressurized air via pneumatic fittings (970) will eventually reach shafts (920) of the sampling heads (900) forming part of fluid communication pathways (644, 654). The pressurized air may flow through these reagent collection shafts (920) and the other fluid communication components that are downstream of these reagent collection shafts (920), eventually exiting shafts (920) of sampling heads (900) forming part of fluid communication pathway (634). The pressurized air may flow for any suitable duration to achieve a desired state of dryness.

Once the drying has been completed, the controller (e.g., controller (121, 512, 522, 562)) may determine whether there are additional wells (782) from which to draw reagents, as represented by block (1014) of FIG. 23. If there are additional wells (782) from which to draw reagents, the process may then provide positioning of sampling head assemblies (750) over the next set of targeted wells (782), as shown in block (1000) of FIG. 23. The above-described stages represented by blocks (1000, 1002, 1004, 1006, 1008, 1010, 1012, 1014) may be reiterated until there are no longer any additional wells (782) from which to draw reagents.

Once there are no longer any additional wells (782) from which to draw reagents, the process may alert the user that all reagents have been used, as represented by block (1016) of FIG. 23. In some versions, this alert may include an audible alert such as a beep or other audible notification. In addition, or in the alternative, this alert may include a visual alert such as an illuminated light, a graphical and/or textual message on a user interface (e.g., user interface (123)), or other visual notification. In versions where system (500, 550, 600) is coupled with a network, the alert may include a text message, email message, or other kind of message conveyed over the network to the user. Alternatively, any other suitable kind(s) of user alert(s) may be provided. In some version, a user alert is omitted. The user alert represented by block (1016) of FIG. 23 is thus optional.

After the foregoing stages has been completed, the user may retrieve the fluid containing encapsulated mRNA and perform testing to determine suitability of the encapsulated mRNA, as represented by block (1018) of FIG. 23. In the context of the arrangement shown in FIG. 8, this may include removing tray (630) from tray support platform (620) and then retrieving the fluid containing encapsulated mRNA from wells (632). In some other versions, the fluid containing encapsulated mRNA is retrieved from wells (632) before tray (630) is removed from tray support platform (620). While fluid processing assembly (700) is configured to deposit the fluid containing encapsulated mRNA in wells (782) in the present example, other variations may deposit the fluid containing encapsulated mRNA in other kinds of containers (e.g., vials, etc.).

As part of the analysis represented by block (1018) of FIG. 23, the user may analyze the fluid containing encapsulated mRNA to determine various properties of the encapsulated mRNA and the fluid in which the mRNA is contained. Such properties may include, but are not necessarily limited to, encapsulation rate, particle size, particle size distribution, zeta potential, in-vitro bioactivity, in-vivo bioactivity, biodistribution in an animal (e.g., in a targeted organ), toxicity, stability, etc. In some variations, fluid processing system (700) and/or other components of system (500, 550, 600) includes one or more integral features that are operable to perform at least some analysis on the fluid containing encapsulated mRNA. For instance, an instrument (510, 560) may include a dynamic light scattering stage that is operable to detect particle size and particle distribution in the fluid containing encapsulated mRNA.

In versions where instrument (510, 560) and/or other components of system (500, 550, 600) includes one or more features that may perform automated analysis of the fluid containing encapsulated mRNA, system (500, 550, 600) may be further configured to provide real-time adjustments to delivery of reagents to process chip (516, 566, 612) in response to results of such testing. In other words, the integrated testing features may be used to provide a feedback loop that allows a controller (512, 522, 562) of system (500, 550, 600) to attempt to refine the formulation process to yield more desirable results.

In some scenarios, a system (500, 550, 600) such as those described above may be operable to execute the above process and yield 96 discrete samples of fluid containing encapsulated mRNA in wells (782) in a tray (780) in less than two hours. In some instances, this overall processing time may be substantially faster than the processing time that might otherwise be needed to yield a similar number of samples of fluid containing encapsulated mRNA using a system like system (100), without an adjunct fluid processing assembly (524, 570, 614, 700).

In the present example, all the wells (782) that contain reagents contain the same formulation of reagents, such that the above-described process may be used to perform 96 tests of the same formulation process using the same formulation inputs. In some other versions, different wells (782) contain different formulations of reagents, such that these different formulations may be tested through the process described above. While trays (780) of the present example each have 96 wells (782), trays (780) may instead have more or fewer than 96 wells (782). While systems (500, 550, 600) are described above in the context of performing screening for mRNA formulation processes, systems (500, 550, 600) may be used in any other suitable kinds of processes. Another example of a process in which systems (500, 550, 600) may be used is described in detail below.

### H. Alternative Example of Method of Utilizing Automated Fluid Delivery System

In some instances, it may be desirable to use a system such any of the various systems (500, 550, 600) described above to perform a plurality of IVT processes. As noted above, an IVT process may include combining a template with reagents such as NTPs (including ATP, CTP, GTP, UTP, modified UTP, and combinations thereof), polymerase, and buffer. Such different IVT processes may be performed in accordance with the above description of block (320) of FIG. 4. Performing IVT processes using any of the versions of systems (500, 550, 600) described herein may facilitate testing of different templates to determine which template provides an RNA solution with the most preferred characteristics.

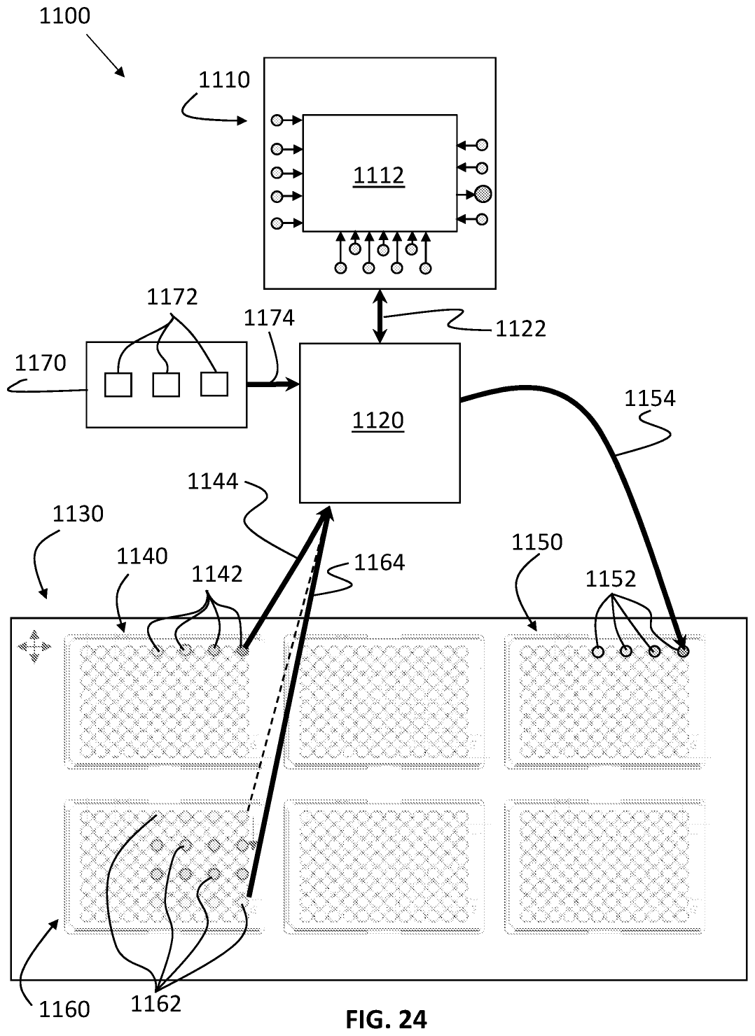

FIG. 24 shows a system (1100) that may be used to perform several IVT processes. System (1100) of this example includes a first fluid processing assembly (1110), a process chip (1112), and a second fluid processing assembly (1120). First fluid processing assembly (1110) may be configured and operable like fluid processing assemblies (514, 564, 610). Process chip (1112) may be configured and operable like any of the variations of process chip (111, 200, 612) described herein. Second fluid processing assembly (1120) may be configured and operable like fluid processing assemblies (524, 570, 614, 700). Fluid processing assemblies (1110, 1120) may be integrated into a single instrument (e.g., similar to system (550)); or provided separately and coupled together (e.g., similar to system (500)). In the present example, process chip (1112) is seated within fluid processing assembly (1110); and fluid processing assemblies (1110, 1120) are in fluid communication with each other via a fluid communication pathway (1122). While a single line is used to represent fluid communication pathway (1122) in FIG. 24, it should be understood that fluid communication pathway (1122) may include several different tubes or other conduits for fluid communication.

Process chip (1112) of the present example is configured to perform IVT processes. By way of example only, process chip (1112) may be configured and operable like process chip (200) described above. Moreover, fluid processing assembly (1110) and process chip (1112) may cooperate to perform IVT processes in accordance with at least some of the teachings of U.S. Pub. No. 2021/0039106, entitled “Microfluidic Apparatus and Methods of Use Thereof,” published Feb. 11, 2021, the disclosure of which is incorporated by reference herein, in its entirety; and/or U.S. Pub. No. 2021/0040472, entitled “Methods and Apparatuses for Manufacturing for Removing Material from a Therapeutic Composition,” published Feb. 11, 2021, the disclosure of which is incorporated by reference herein, in its entirety. The templates and reagents that are used to perform the IVT processes on fluid processing assembly (1110) and process chip (1112) may be provided via fluid processing assembly (1120) as described below.

System (1100) of this example further includes a tray support platform (1130), with a plurality of trays (1140, 1150, 1160) arranged in a grid on platform (1130). Tray support platform (1130) may be configured and operable like tray support platforms (620, 770) described above; and trays (1140, 1150, 1160) may be configured and operable like trays (630, 640, 650, 660, 670, 680, 780) described above. Thus, in the present example, each tray (1140, 1150, 1160) defines a plurality of wells (1142, 1152, 1162). Each well (1142, 1152, 1162) is configured to hold a volume of fluid. Fluid processing assembly (1120) includes a plurality of fluid communication pathways (1144, 1164, 1154) that are configured to provide communication of fluid from and/or to wells (1142, 1152, 1162). Fluid processing assembly (1120) may be operated such that fluid communication pathways (1144, 1164, 1154) move in relation to trays (1140, 1150, 1160), as described above with in the context of fluid processing assembly (700) to selectively communicate with wells (1142, 1152, 1162). As also described above in the context of fluid processing assembly (700), tray support platform (1130) may also move in relation to fluid communication pathways (1142, 1152, 1162) to enable fluid communication pathways (1144, 1164, 1154) to reach different wells (1142, 1152, 1162).

In the present example, tray (1140) is used to hold a plurality of different templates. In other words, each well (1142) may contain a different template. By way of example only, the different templates may include approximately 2,000 base pairs of nucleotides encoding luciferase; or approximately 1,000 base pairs encoding green-fluorescent protein (GFP). Alternatively, any other suitable templates may be used. Tray (1150) is used to hold fluid output from fluid processing assembly (1110), which may include different mRNA solutions generated via IVT processes utilizing different templates from tray (1140); and waste (e.g., fluids generated while cleaning and rinsing fluid processing assemblies (1110, 1120) as described below). In other words, some wells (1152) may contain different mRNA solutions corresponding to a certain templates from corresponding wells (1142); and some other wells (1152) may contain waste. In some other variations, waste is communicated to one or more other receptacles (i.e., not to tray (1140).

Tray (1160) is used to contain different cleaning agents and rinsing agents. In other words, some wells (1162) may contain a cleaning agent and some other wells (1162) may contain a rinsing agent. By way of example only, such cleaning agents may include tris(hydroxymethyl)aminomethane (“tris”), sodium hydroxide, sodium hypochlorite, polysorbate, water, and combinations thereof; and such rising agents may include water. Alternatively, any other suitable cleaning agents and rinsing agents may be used.

In the present example, a vial assembly (1170) is also coupled with second fluid processing assembly (1120) via a fluid communication pathway (1174) to form part of system (1100). Vial assembly (1170) includes a plurality of vials (1172) containing other reagents that are used during the different IVT processes performed using system (1100). By way of example only, the reagents in vials (1172) may include NTPs (including ATP, CTP, GTP, UTP, modified UTP, and combinations thereof), polymerase, and/or buffer. Alternatively, any other suitable reagents may be used. In the present example, the same reagents from vials (1172) are used throughout all the IVT processes; even though different templates from different wells (1142) are used throughout all the IVT processes. In other words, in the present example, only different templates are used in the different IVT processes performed using system (1100). In some other variations, different reagents are used, in addition to different templates, in the different IVT processes performed using system (1100). In some such variations, the different reagents are contained in different respective wells in one or more trays on tray support platform (1130).

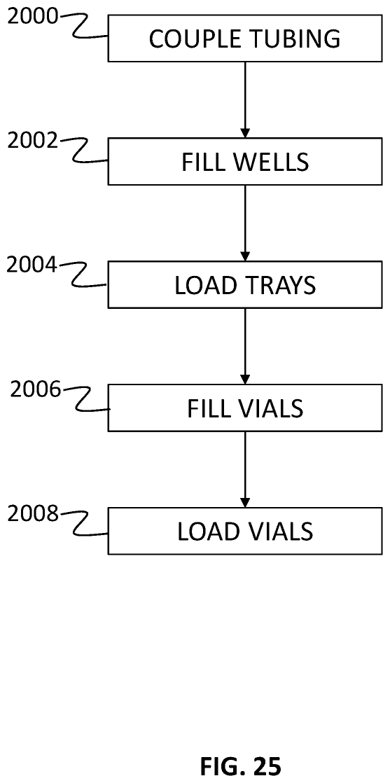

FIG. 25 shows an example of a process that may be used to set up system (1100) for use. While the example of FIG. 25 is provided in the context of system (1100), the same kind of process may be utilized to set up any of the various other systems described herein. It should also be understood that the steps shown in FIG. 25 and described below may be performed in parallel and in any other suitable sequence, such that the process need not necessarily follow the particular sequence depicted in FIG. 25. As an example of a first step for setting up system (1100), and as shown in block (2000), tubing may be coupled between fluid processing assemblies (1110, 1120); and between fluid vial assembly (1170) and fluid processing assembly (1120). Such tubing may establish fluid communication pathways (1122, 1174) as described above. Next, as shown in block (2002), wells (1142) are filled with different templates; and wells (1162) area filled with cleaning agents and rinsing agents. Trays (1140, 1150, 1160) are then loaded onto platform (1130), as shown in block (2004). Vials (1172) are filled with reagents, as shown in block (2206); and then vials (1172) are loaded into fluid vial assembly (1170), as shown in block (2208).

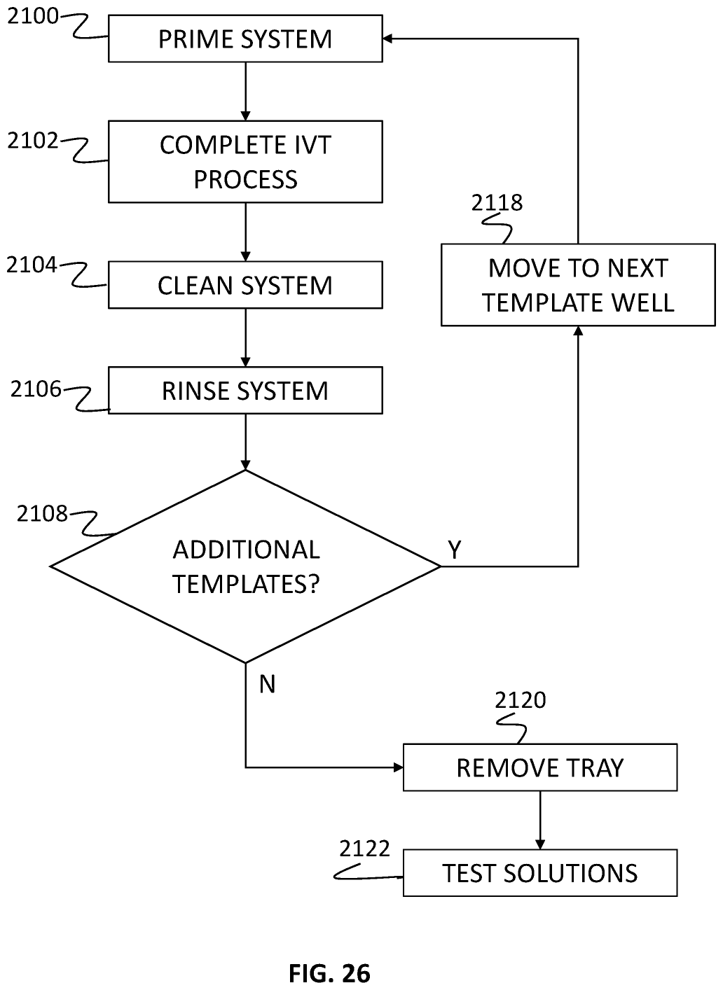

FIG. 26 shows an example of a process that may be performed after system (1100) has been set up for use. As shown in block (2100), system (1110) may first be primed. This priming may include communication of fluids from one or more vials (1172) and wells (1142) through fluid processing assemblies (1110, 1120) until the fluids reach predetermined positions within channels of process chip (1112). By way of example only, such priming may be carried out in accordance with at least some of the teachings of International Patent App. No. PCT/US2022/031277, entitled “Systems and Methods to Detect Presence of Fluids,” filed May 27, 2022, the disclosure of which is incorporated by reference herein, in its entirety. In some such versions, a camera is used to monitor the predetermined positions within channels of process chip (1112); and image processing techniques are used to determine when fluids reach those predetermined positions. The flow of a fluid may be automatically stopped in response to the determination that the fluid has reached its corresponding predetermined position within the corresponding channel of process chip (1112). Such an automated priming process may be carried out via a controller that is configured and operable like any of the controllers (121, 512, 522, 562) described herein. It should be understood that, in some versions of this initial priming process, just one single template may be communicated to process chip (1112) from a corresponding single well (1142).

After system (1110) has been sufficiently primed with the first template, an IVT process may be carried out on process chip (1112) using that first template, as shown in block (2102). Part of this IVT process includes communication of the resulting mRNA solution to a selected well (1152) of tray (1150). Once that first IVT process is completed, and as shown in block (2104), fluid processing assemblies (1110, 1120) may be driven to communicate a cleaning agent through the same fluid lines that were used in fluid processing assemblies (1110, 1120) and process chip (1112) during the IVT process. In some versions, the cleaning agent is only communicated through the fluid line(s) through which the first template and the first resulting mRNA solution were communicated. In versions where process chip (1112) has a plurality of fluid chambers, the cleaning agent may be communicated to two or more fluid chambers simultaneously; or to two or more fluid chambers in a sequence. The cleaning agent may be taken from one or more of wells (1162). In some versions, more than one cleaning agent is used. In such versions, the different cleaning agents may come from different wells (1162). The waste generated through the cleaning process (block (2104)) may be communicated to one or more wells (1142) or some other waste receptacle.

Once the cleaning agent(s) has/have been sufficiently communicated through the appropriate fluid lines, and as shown in block (2106), fluid processing assemblies (1110, 1120) may be driven to communicate a rinsing agent through the same fluid lines that were used in fluid processing assemblies (1110, 1120) and process chip (1112) during the IVT process. In some versions, the rinsing agent is only communicated through the fluid line(s) through which the first template and the first resulting mRNA solution were communicated. In versions where process chip (1112) has a plurality of fluid chambers, the rinsing agent may be communicated to two or more fluid chambers simultaneously; or to two or more fluid chambers in a sequence. The rinsing agent may be taken from one or more of wells (1162). In some versions, more than one rinsing agent is used. In such versions, the different rinsing agents may come from different wells (1162). The waste generated through the rinsing process (block (2106)) may be communicated to one or more wells (1142) or some other waste receptacle.

Once the rinsing agent(s) has/have been sufficiently communicated through the appropriate fluid lines, and as shown in block (2108), a determination is made as to whether an additional template should be used in a subsequent IVT process. Such a determination may be made via a controller that is configured and operable like any of the controllers (121, 512, 522, 562) described herein. If another template is available for use in another IVT process, then fluid processing assembly (1120) may establish fluid communication with the corresponding well (1142) as shown in block (2118). Fluid processing assembly (1120) may then again prime system (1100) with that other template and any other reagents, as described above with reference to block (2100). The steps described above with reference to blocks (2102, 2104, 2106, 2108, 2118) may be repeated until the last template in the last filled will (1142) has been used.

Once there are no more templates to use, the operator may remove tray (1150) from tray support platform (1130), as shown in block (2110). The operator may then perform tests on the mRNA solutions contained in wells (1152), to determine which mRNA solution provides the most desired characteristics, as shown in block (2122). Once the most preferred mRNA solution is identified, the operator may correlate the preferred mRNA solution with the template that was used to form the preferred mRNA solution; and then later use the same template in IVT processes to generate more mRNA solution with the preferred characteristics.

In some versions, the removal of tray (1150) from tray support platform (1130) (block (2120)) and the testing of the solutions in wells (1152) (block (2122)) are performed by a human operator. In some other versions, the removal of tray (1150) from tray support platform (1130) (block (2120)) and/or the testing of the solutions in wells (1152) (block (2122)) are automated. For instance, a robotic system may be used to remove tray (1150) from tray support platform (1130). In addition, or in the alternative, a robotic system may be used to test the solutions in wells (1152). Any suitable techniques may be used to test the solutions in wells (1152).

The foregoing example refers to a single IVT process being performed per each different kind of template. However, it should be understood that several IVT processes may be run for each template, such that each template may be effectively tested through a panel of several corresponding mRNA solution samples. In other words, the process described above with reference to FIG. 26 may be carried out several times with a single kind of template to generate several samples of mRNA solutions generated using that single kind of template. The same process may again be repeated several times using another kind of template such that the end result may include several different panels of mRNA solution samples, with each panel of mRNA solution samples corresponding to a different respective template.

### VI. Miscellaneous

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these implementations may be readily apparent to those skilled in the art, and generic principles defined herein may be applied to other implementations. Thus, many changes and modifications may be made to the subject technology, by one having ordinary skill in the art, without departing from the scope of the subject technology. For instance, different numbers of a given module or unit may be employed, a different type or types of a given module or unit may be employed, a given module or unit may be added, or a given module or unit may be omitted.

Some versions of the examples described herein may be implemented using a processor, which may be part of a computer system and communicate with a number of peripheral devices via bus subsystem. Versions of the examples described herein that are implemented using a computer system may be implemented using a general-purpose computer that is programmed to perform the methods described herein. Alternatively, versions of the examples described herein that are implemented using a computer system may be implemented using a specific-purpose computer that is constructed with hardware arranged to perform the methods described herein. Versions of the examples described herein may also be implemented using a combination of at least one general-purpose computer and at least one specific-purpose computer.

In versions implemented using a computer system, each processor may include a central processing unit (CPU) of a computer system, a microprocessor, an application-specific integrated circuit (ASIC), other kinds of hardware components, and combinations thereof. A computer system may include more than one type of processor. The peripheral devices of a computer system may include a storage subsystem including, for example, memory devices and a file storage subsystem, user interface input devices, user interface output devices, and a network interface subsystem. The input and output devices may allow user interaction with the computer system. The network interface subsystem may provide an interface to outside networks, including an interface to corresponding interface devices in other computer systems. User interface input devices may include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system.

In versions implemented using a computer system, a user interface output device may include a display subsystem, a printer, a fax machine, or non-visual displays such as audio output devices. The display subsystem may include a cathode ray tube (CRT), a flat-panel device such as a liquid crystal display (LCD), a projection device, or some other mechanism for creating a visible image. The display subsystem may also provide a non-visual display such as audio output devices. In general, use of the term “output device” is intended to include all possible types of devices and ways to output information from computer system to the user or to another machine or computer system.

In versions implemented using a computer system, a storage subsystem may store programming and data constructs that provide the functionality of some or all of the modules and methods described herein. These software modules may be generally executed by the processor of the computer system alone or in combination with other processors. Memory used in the storage subsystem may include a number of memories including a main random-access memory (RAM) for storage of instructions and data during program execution and a read only memory (ROM) in which fixed instructions are stored. A file storage subsystem may provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem in the storage subsystem, or in other machines accessible by the processor.

In versions implemented using a computer system, the computer system itself may be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the example of the computer system described herein is intended only as a specific example for purposes of illustrating the technology disclosed. Many other configurations of a computer system are possible having more or fewer components than the computer system described herein.

As an article of manufacture, rather than a method, a non-transitory computer readable medium (CRM) may be loaded with program instructions executable by a processor. The program instructions when executed, implement one or more of the computer-implemented methods described above. Alternatively, the program instructions may be loaded on a non-transitory CRM and, when combined with appropriate hardware, become a component of one or more of the computer-implemented systems that practice the methods disclosed.

Underlined and/or italicized headings and subheadings are used for convenience only, do not limit the subject technology, and are not referred to in connection with the interpretation of the description of the subject technology. All structural and functional equivalents to the elements of the various implementations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

1-85. (canceled)

86. A method comprising:
communicating a first template to a process chip, the first template being located in a first well of a first tray;
communicating a first set of one or more reagents to the process chip;
performing in vitro transcription (IVT) utilizing the first template and the first set of one or more reagents, thereby creating a first RNA solution;
communicating the first RNA solution to a first well of a second tray;
communicating a second template to the process chip, the second template being located in a second well of the first tray;
communicating a second set of one or more reagents to the process chip;
performing in vitro transcription (IVT) utilizing the second template and the second set of one or more reagents, thereby creating a second RNA solution; and
communicating the second RNA solution to a second well of the second tray.

87. The method of claim 86, the one or more reagents being contained in one or more corresponding vials.

88. The method of claim 86, further comprising communicating a cleaning agent through the process chip between the act of communicating the first RNA solution to the first well of the second tray and communicating the second template to the process chip.

89. The method of claim 88, the cleaning agent comprising a component selected from the group consisting of tris(hydroxymethyl)aminomethane, sodium hydroxide, sodium hypochlorite, polysorbate, water, and combinations thereof.

90. The method of claim 88, the cleaning agent being communicated from one or more wells of a third tray.

91. The method of claim 86, further comprising communicating a rinsing agent through the process chip between the act of communicating the first RNA solution to the first well of the second tray and communicating the second template to the process chip.

92. The method of claim 91, the rinsing agent comprising water.

93. The method of claim 91, the rinsing agent being communicated from one or more wells of a third tray.

94. The method of claim 86, further comprising automatically priming the process chip with the first template and the first set of one or more reagents.

95. The method of claim 86, the first set of one or more reagents comprising one or more components selected from the group of NTPs, polymerase, and buffer.

96. The method of claim 86, the first template comprising luciferase or green-fluorescent protein.

97. The method of claim 86, further comprising:
testing the first RNA solution;
testing the second RNA solution; and
determining which of the first RNA solution and the second RNA solution provides a best fit with one or more criteria.

98. The method of claim 97, further comprising transporting the second tray to a testing location to perform the acts of testing the first RNA solution and testing the second RNA solution.

99. A processor-readable medium including contents that are configured to cause a processor to process data by performing the method of claim 86.

100. A system comprising:
a first fluid processing assembly, the first fluid processing assembly comprising a process chip;
a second fluid processing assembly, the second fluid processing assembly comprising a first tray and a second tray;
one or more controllers, the one or more controllers being configured to execute a control algorithm to automatically perform the following:
communicate a first template to the process chip, the first template being located in a first well of the first tray,
communicate a first set of one or more reagents to the process chip,
perform in vitro transcription (IVT) utilizing the first template and the first set of one or more reagents, thereby creating a first RNA solution,
communicate the first RNA solution to a first well of the second tray,
communicate a second template to the process chip, the second template being located in a second well of the first tray,
communicate a second set of one or more reagents to the process chip,
perform in vitro transcription (IVT) utilizing the second template and the second set of one or more reagents, thereby creating a second RNA solution, and
communicate the second RNA solution to a second well of the second tray.
